(19) United States
(12) Patent Application Publication
(10) Pub. No.: US 2024/0112560 A1
(43) Pub. Date: Apr. 4, 2024

(54) PREVENTION OF FALL EVENTS USING INTERVENTIONS BASED ON DATA ANALYTICS

(71) Applicant: Scanalytics, Inc., Milwaukee, WI (US)

(72) Inventor: JOSEPH SCANLIN, Milwaukee, WI (US)

(73) Assignee: Scanalytics, Inc., Milwaukee, WI (US)

(21) Appl. No.: 18/541,803

(22) Filed: Dec. 15, 2023

Related U.S. Application Data
(63) Continuation-in-part of application No. 17/116,582, filed on Dec. 9, 2020.
Application No. 17/116,582 is a continuation-in-part of application No. 16/696,802, filed on Nov. 26, 2019, now Pat. No. 10,954,677.
(60) Provisional application No. 62/956,532, filed on Jan. 2, 2020.

(51) Int. Cl. G08B 21/04; A61B 5/11; G06V 10/764; G16H 10/60

(52) U.S. Cl. CPC G08B 21/043; A61B 5/1118; A61B 5/112; G06V 10/764; G16H 10/60

(57) ABSTRACT

In some embodiments, a method is disclosed for determining a propensity for a fall event to occur. The method may include receiving data from a sensing device in a smart floor tile, monitoring a parameter pertaining to a gait of a person based on the data, determining an amount of gait deterioration based on the parameter, and determining whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.

### CROSS-REFERENCE TO RELATED APPLICATIONS

The present application is a continuation-in-part of U.S. Non-Provisional application Ser. No. 17/116,582, titled “PATH ANALYTICS OF PEOPLE IN A PHYSICAL SPACE USING SMART FLOOR TILES” filed Dec. 9, 2020, which is a continuation-in-part of U.S. Non-Provisional application Ser. No. 16/696,802, titled “CONNECTED MOULDING FOR USE IN SMART BUILDING CONTROL”, filed Nov. 26, 2019. The present application further claims priority to and the benefit of U.S. Provisional Patent Application No. 62/956,532, titled “PREVENTION OF FALL EVENTS USING INTERVENTIONS BASED ON DATA ANALYTICS”, filed Jan. 2, 2020. The content of these applications are incorporated herein by reference in their entirety for all purposes.

### TECHNICAL FIELD

This disclosure relates to data analytics. More specifically, this disclosure relates to prevention of fall events using interventions based on data analytics.

### BACKGROUND

Fall events present a public health concern, especially among older people, and are related to morbidity and mortality. Studies have shown a significant percentage of people over 65 fall each year. The percentage increases for older people in care homes. The outcome of fall events may include impacts on social and community care. The social impacts may include fear of falling that influences the quality of life of the patient and increases social isolation. There are certain environmental hazards that increase the chance of fall events occurring, such as wet floors, poor lighting, lack of bedrails, improper bed height, low nurse staffing, and the like. There are also certain physical characteristics tied to gait, balance, and/or neurological conditions of a person that are risks for causing a fall event for the person. Reducing the number of fall events may improve a quality of life of a person, allow the person to be active longer, and in some instances, save lives.

### SUMMARY

In one embodiment, a method for determining a propensity for a fall event to occur includes receiving data from a sensing device in a smart floor tile, monitoring a parameter pertaining to a gait of a person based on the data, determining an amount of gait deterioration based on the parameter, and determining whether the propensity for the fall event for the person satisfies a threshold propensity condition based on (i) the amount of gait deterioration satisfying a threshold deterioration condition, (ii) the amount of gait deterioration satisfying the threshold deterioration condition within a threshold time period, or some combination thereof.

In one embodiment, a tangible, non-transitory computer-readable medium stores instructions that, when executed, cause a processing device to receive data from a sensing device in a smart floor tile, monitor a parameter pertaining to a gait of a person based on the data, determine an amount of gait deterioration based on the parameter, and determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.

In one embodiment, a system includes a memory device storing instructions and a processing device communicatively coupled to the memory device. The processing device executes the instructions to receive data from a sensing device in a smart floor tile, monitor a parameter pertaining to a gait of a person based on the data, determine an amount of gait deterioration based on the parameter, and determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIGS. 1A-1E illustrate various example configurations of components of a system according to certain embodiments of this disclosure;

FIG. 2 illustrates an example component diagram of a moulding section according to certain embodiments of this disclosure;

FIG. 3 illustrates an example backside view of a moulding section according to certain embodiments of this disclosure;

FIG. 4 illustrates a network and processing context for smart building control using directional occupancy sensing and fall prediction/prevention 4

according to certain embodiments of this disclosure;

FIG. 5 illustrates aspects of a smart floor tile according to certain embodiments of this disclosure;

FIG. 6 illustrates a master control device according to certain embodiments of this disclosure;

FIG. 7A illustrate an example of a method for predicting a fall event according to certain embodiments of this disclosure;

FIG. 7B illustrates an example architecture including machine learning models to perform the method of FIG. 7A according to certain embodiments of this disclosure;

FIG. 8 illustrates example interventions according to certain embodiments of this disclosure;

FIG. 9 illustrates example parameters that may be monitored according to certain embodiments of this disclosure;

FIG. 10 illustrates an example of a method for using gait baseline parameters to determine an amount of gait deterioration according to certain embodiments of this disclosure;

FIG. 11 illustrates an example of a method for subtracting data associated with certain people from gait analysis according to certain embodiments of this disclosure;

FIG. 12A-B illustrate an overhead view of an example for subtracting data associated with certain people from gait analysis according to certain embodiments of this disclosure; and

FIG. 13 illustrates an example computer system according to embodiments of this disclosure.

### NOTATION AND NOMENCLATURE

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer or section from another region, layer or section. Terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), solid state drives (SSDs), flash memory, or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

The term “moulding” may be spelled as “molding” herein.

The term “fall event” may refer to a person falling by moving downward from a higher to a lower level. The movement may be rapid and freely without control.

### DETAILED DESCRIPTION

The following discussion is directed to various embodiments of the disclosed subject matter. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

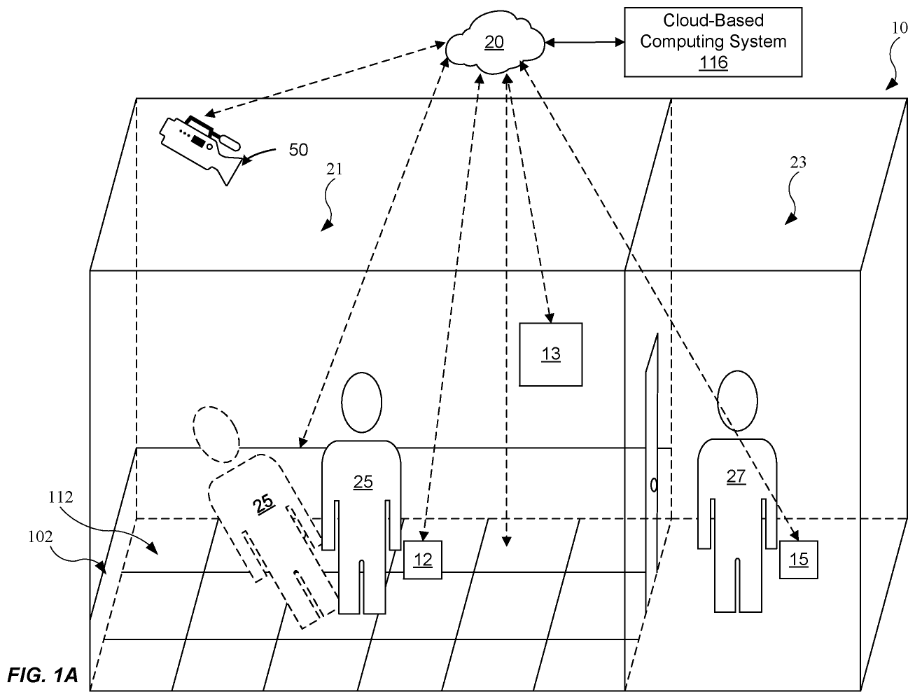

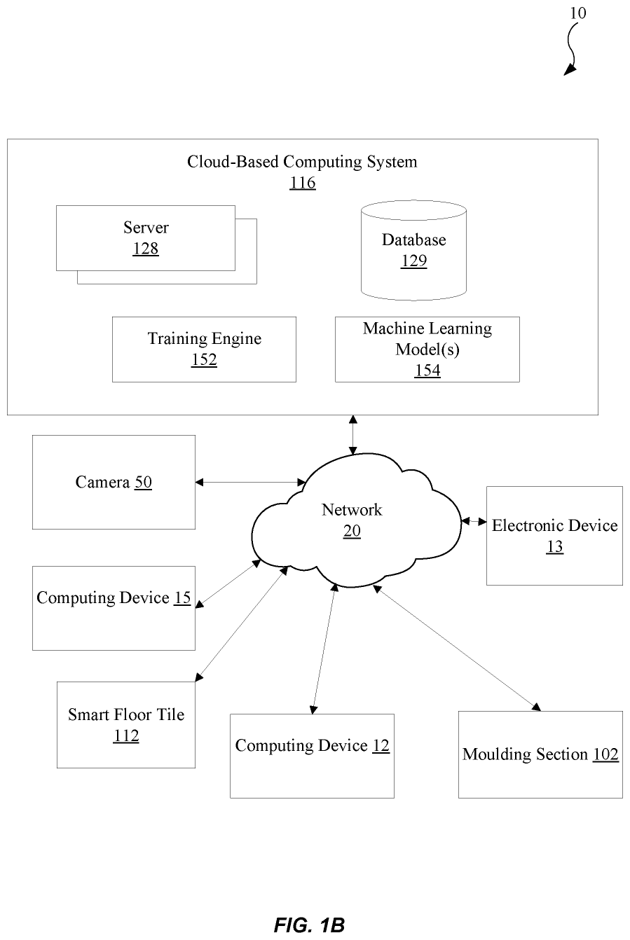

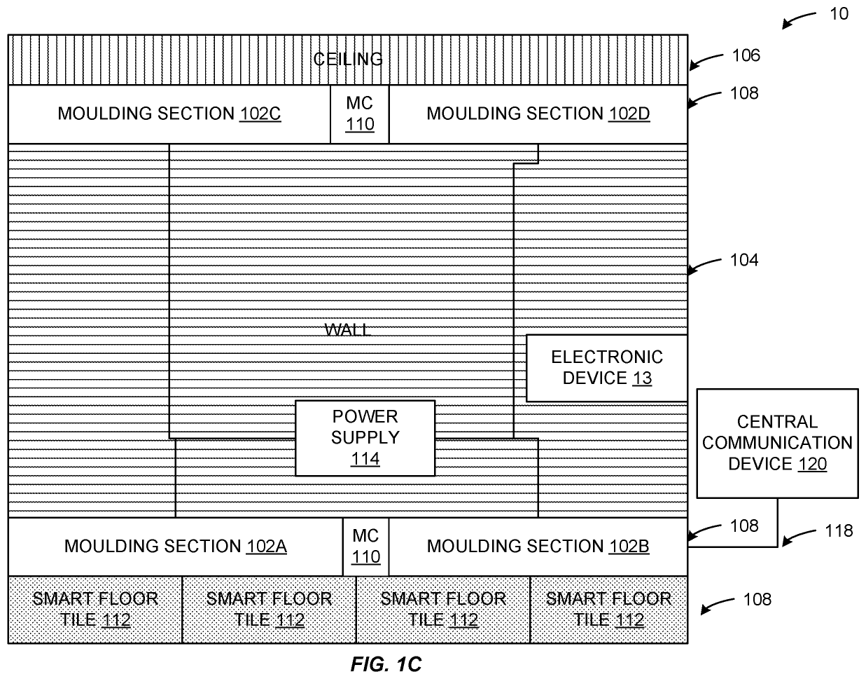

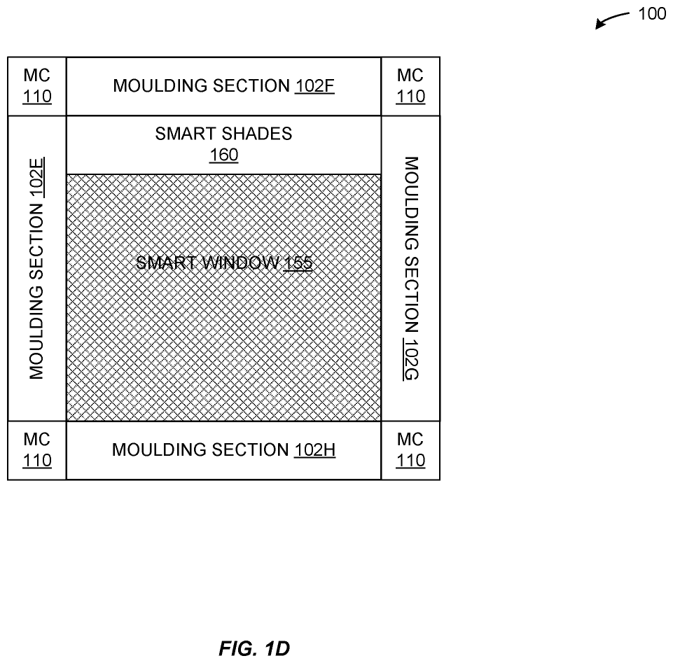

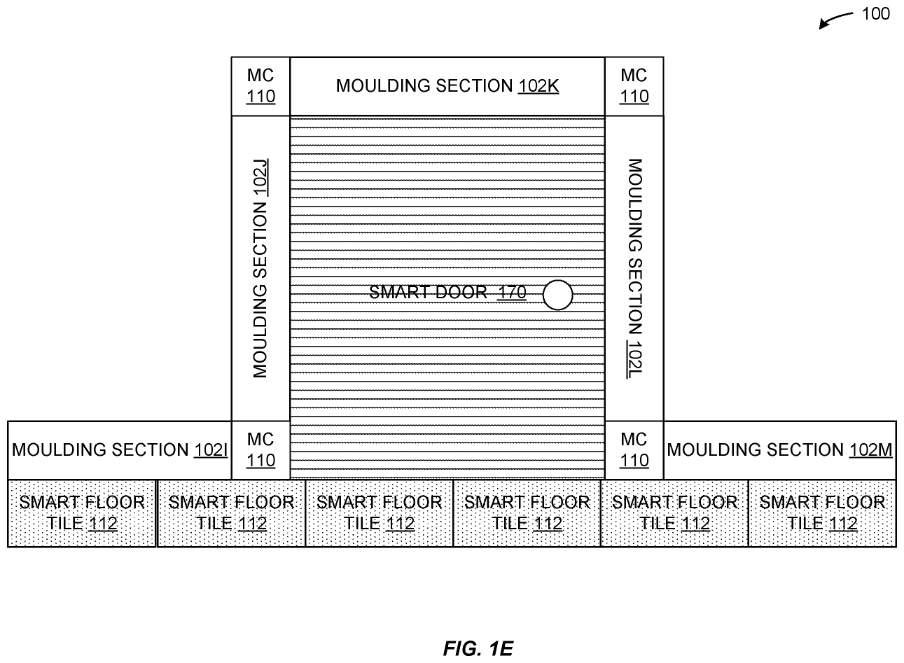

FIGS. 1A through 13, discussed below, and the various embodiments used to describe the principles of this disclosure in this patent document are by way of illustration only and should not be construed in any way to limit the scope of the disclosure.

Embodiments as disclosed herein relate to prevention of fall events using interventions based on data analytics. People typically experience fall events as they move from a first location to a second location by performing a physical activity, such as walking, jumping, jogging, and/or running. Research shows that the propensity for a fall event to occur increases as people age. This is due to aging being generally associated with decrease in muscle strength and muscle mass that may result in reduced functional capacity physical frailty, impaired mobility, and/or accidental falls. There are numerous risks that may increase the propensity for the fall event to occur. For example, the risks may include characteristics of a gait and/or balance of the person, physical measurements of the person, medical history, fracture history, fall history, urinary incontinence, neurological conditions, medication, and the like. As the number of risks that a person is exposed to increases, the propensity for the fall event may increase.

It is desired to reduce the number of fall events from occurring to improve the quality of life of people and/or extend the lifespan of people. The disclosed embodiments generally relate to predicting that a fall event is imminent or going to occur in the future and performing an intervention to prevent the fall event from occurring. The embodiments may be used in any suitable location where people move around, such as a home, a mall, an office, and/or any suitable space. In particular, the embodiments may be beneficial in care facilities, such as nursing homes, where elderly people reside or are staying for a period of time, as elderly people are more inclined to experience fall events. Reducing the fall events from occurring may be physically and socially beneficial to people. Further, reducing the fall events may be associated with insurance companies reducing expenses by paying for fewer claims associated with fall events at the care facilities. In turn, the insurance companies may reduce interest rates and/or fees that the medical facilities pay for coverage.

To predict and/or prevent the fall events from occurring, some embodiments of the present disclosure may utilize smart floor tiles that are disposed in a physical space where a person is located. For example, the smart floor tiles may be installed in a floor of a room of a care facility where an elderly person receives care. The smart floor tiles may be capable of measuring data (e.g., pressure) associated with footsteps of the person and transmitting the measured data to a cloud-based computing system that analyzes the measured data. In some embodiments, moulding sections and/or a camera may be used to measure the data and/or supplement the data measured by the smart floor tiles. The accuracy of the measurements pertaining to the gait and/or balance of the person may be improved using the smart floor tiles as they measure the physical pressure of the footsteps of the person to track the path of the person and other gait characteristics (e.g., width of feet, speed of gait, etc.).

Barring unforeseeable changes in human locomotion, humans can be expected to generate measurable interactions with buildings through their footsteps on buildings' floors. Embodiments according to the present disclosure use the measured data from the smart floor tiles to predict and/or prevent fall events from occurring. Further, in some embodiments the smart floor tiles may help realize the potential of a “smart building” by providing, amongst other things, control inputs for a building's environmental control systems using directional occupancy sensing based on occupants' interaction with building surfaces, including, without limitation, floors, and/or interaction with a physical space including their location relative to moulding sections.

The moulding sections, may include a crown moulding, a baseboard, a shoe moulding, a door casing, and/or a window casing, that are located around a perimeter of a physical space. The moulding sections may be modular in nature in that the moulding sections may be various different sizes and the moulding sections may be connected with moulding connectors. The moulding connectors may be configured to maintain conductivity between the connected moulding sections. To that end, each moulding section may include various components, such as electrical conductors, sensors, processors, memories, network interfaces, and so forth that enable communicating data, distributing power, obtaining moulding section sensor data, and so forth. The moulding sections may use various sensors to obtain moulding section sensor data including the location of objects in a physical space as the objects move around the physical space. The moulding sections may use moulding section sensor data to determine a path of the object in the physical space and/or to control other electronic devices (e.g., smart shades, smart windows, smart doors, HVAC system, smart lights, and so forth) in the smart building. Accordingly, the moulding sections may be in wired and/or wireless communication with the other electronic devices. Further, the moulding sections may be in electrical communication with a power supply. The moulding sections may be powered by the power supply and may distribute power to smart floor tiles that may also be in electrical communication with the moulding sections.

The camera may provide a livestream of video data and/or image data to the cloud-based computing system. The data from the camera may be used to identify certain people in a room and/or track the path of the people in the room. Further, the data may be used to monitor one or more parameters pertaining to a gait of the person to aid in predicting and/or preventing fall events.

The cloud-based computing system may monitor one or more parameters of the person based on the measured data from the smart floor tiles, the moulding sections, and/or the camera. The one or more parameters may be associated with the gait of the person and/or the balance of the person. There are numerous other parameters associated with the person that may be monitored, as described in further detail below.

Based on the one or more parameters, the cloud-based computing system may determine an amount of gait deterioration. For example, the cloud-based computing may determine that the speed of the gait of the person reduced by a certain amount, and the amount of gait deterioration is a certain percentage or value based on the amount of gait speed reduction. The cloud-based computing system may determine whether a propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period. The propensity of the fall event may be scored or categorized into a level of 1 to 5 (any suitable range), where a 1 is the lowest score or category where the propensity for the fall event is the lowest and not likely to occur and a 5 is the highest score or category where the propensity for the fall event is the highest and most likely to occur. The cloud-based computing system may use one or more machine learning models trained to monitor the parameter pertaining to the gait of the person based on the data, determine the amount of gait deterioration based on the parameter, and/or determine whether the propensity for the fall event for the person satisfies the threshold propensity condition.

If the propensity for the fall event does not satisfy the threshold propensity condition, the cloud-based computing system may continue to monitor the one or more parameters. If the propensity for the fall event satisfies the threshold propensity condition, the cloud-based computing system may determine an intervention to perform based on the propensity for the fall event. For example, if the propensity for the fall event is high (e.g., the amount of gait deterioration was high within a short amount of time), a more severe intervention may be performed. The interventions may include transmitting a message to a computing device of the person and/or a medical personnel (e.g., a nurse in the care facility), causing an alarm to be triggered in the care facility in which the person is located, changing a property of an electronic device located in a physical space with the person, changing a care plan for the person and the like.

Turning now to the figures, FIGS. 1A-1E illustrate various example configurations of components of a system 10 according to certain embodiments of this disclosure. FIG. 1A visually depicts components of the system in a first room 21 and a second room 23 and FIG. 1B depicts a high-level component diagram of the system 10. For purposes of clarity, FIGS. 1A and 1B are discussed together below.

The first room 21, in this example, is a care room in a care facility where a person 25 is being treated. However, the first room 21 may be any suitable room that includes a floor capable of being equipped with smart floor tiles 112, moulding sections 102, and/or a camera 50. The second room 23, in this example, is a nursing station in the care facility.

The person 25 has a computing device 12, which may be a smartphone, a laptop, a tablet, a pager, or any suitable computing device. A medical personnel 27 in the second room 23 also has a computing device 15, which may be a smartphone, a laptop, a tablet, a pager, or any suitable computing device. The first room 21 may also include at least one electronic device 13, which may be any suitable electronic device, such as a smart thermostat, smart vacuum, smart light, smart speaker, smart electrical outlet, smart hub, smart appliance, smart television, etc.

Each of the smart floor tiles 112, moulding sections 102, camera 50, computing device 12, computing device 15, and/or electronic device 13 may be capable of communicating, either wirelessly and/or wired, with a cloud-based computing system 116 via a network 20. As used herein, a cloud-based computing system refers, without limitation, to any remote or distal computing system accessed over a network link. Each of the smart floor tiles 112, moulding sections 102, camera 50, computing device 12, computing device 15, and/or electronic device 13 may include one or more processing devices, memory devices, and/or network interface devices.

The network interface devices of the smart floor tiles 112, moulding sections 102, camera 50, computing device 12, computing device 15, and/or electronic device 13 may enable communication via a wireless protocol for transmitting data over short distances, such as Bluetooth, ZigBee, near field communication (NFC), etc. Additionally, the network interface devices may enable communicating data over long distances, and in one example, the smart floor tiles 112, moulding sections 102, camera 50, computing device 12, computing device 15, and/or electronic device 13 may communicate with the network 20. Network 20 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 12 and/or computing device 15 may be any suitable computing device, such as a laptop, tablet, smartphone, or computer. The The computing device 12 and/or computing device 15 may include a display that is capable of presenting a user interface. The user interface may be implemented in computer instructions stored on a memory of the computing device 12 and/or computing device 15 and executed by a processing device of the computing device 12 and/or computing device 15. The user interface 105 be a stand-alone application that is installed on the computing device 12 and/or computing device 15 or may be an application (e.g., website) that executes via a web browser. The user interface may present various interventions including screens, notifications, and/or messages to the person 25 and/or the medical personnel 27.

For the computing device 12 of the person, the screens, notifications, and/or messages may be received from the cloud-based computing system 116 and may indicate that a fall event is predicted to occur in the future. The screens, notifications, and/or messages may encourage the person 25 to stop walking, to grab onto a supporting structure, to walk slower, or the like. For the computing device 15 of the medical personnel 27, the screens, notifications, and/or messages may be received from the cloud-based computing system 116 and may indicate that a fall event is predicted for the person 25. The screens, notifications, and/or messages may encourage the medical personnel 27 to tend to the person 25 in the first room 21 to attempt to prevent the fall event from occurring.

In some embodiments, the cloud-based computing system 116 may include one or more servers 128 that form a distributed, grid, and/or peer-to-peer (P2P) computing architecture. Each of the servers 128 may include one or more processing devices, memory devices, data storage, and/or network interface devices. The servers 128 may be in communication with one another via any suitable communication protocol. The servers 128 may receive data from the smart floor tiles 112, moulding sections 102, and/or the camera 50 and monitor a parameter pertaining to a gait of the person 25 based on the data. For example, the data may include pressure measurements obtained by a sensing device in the smart floor tile 112. The pressure measurements may be used to accurately track footsteps of the person 25, walking paths of the person 25, gait characteristics of the person 25, walking patterns of the person 25 throughout each day, and the like. The servers 128 may determine an amount of gait deterioration based on the parameter. The servers 128 may determine whether a propensity for a fall event for the person 25 satisfies a threshold propensity condition based on (i) the amount of gait deterioration satisfying a threshold deterioration condition, or (ii) the amount of gait deterioration satisfying the threshold deterioration condition within a threshold time period. If the propensity for the fall event for the person 25 satisfies the threshold propensity condition, the servers 128 may select one or more interventions to perform for the person 25 to prevent the fall event from occurring and may perform the one or more selected interventions. The servers 128 may use one or more machine learning models 154 trained to monitor the parameter pertaining to the gait of the person 25 based on the data, determine the amount of gait deterioration based on the parameter, and/or determine whether the propensity for the fall event for the person satisfies the threshold propensity condition.

In some embodiments, the cloud-based computing system 116 may include a training engine 152 and/or the one or more machine learning models 154. The training engine 152 and/or the one or more machine learning models 154 may be communicatively coupled to the servers 128 or may be included in one of the servers 128. In some embodiments, the training engine 152 and/or the machine learning models 154 may be included in the computing device 12, computing device 15, and/or electronic device 13.

The one or more of machine learning models 154 may refer to model artifacts created by the training engine 152 using training data that includes training inputs and corresponding target outputs (correct answers for respective training inputs). The training engine 152 may find patterns in the training data that map the training input to the target output (the answer to be predicted), and provide the machine learning models 154 that capture these patterns. The set of machine learning models 154 may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and/or fully connected neural networks.

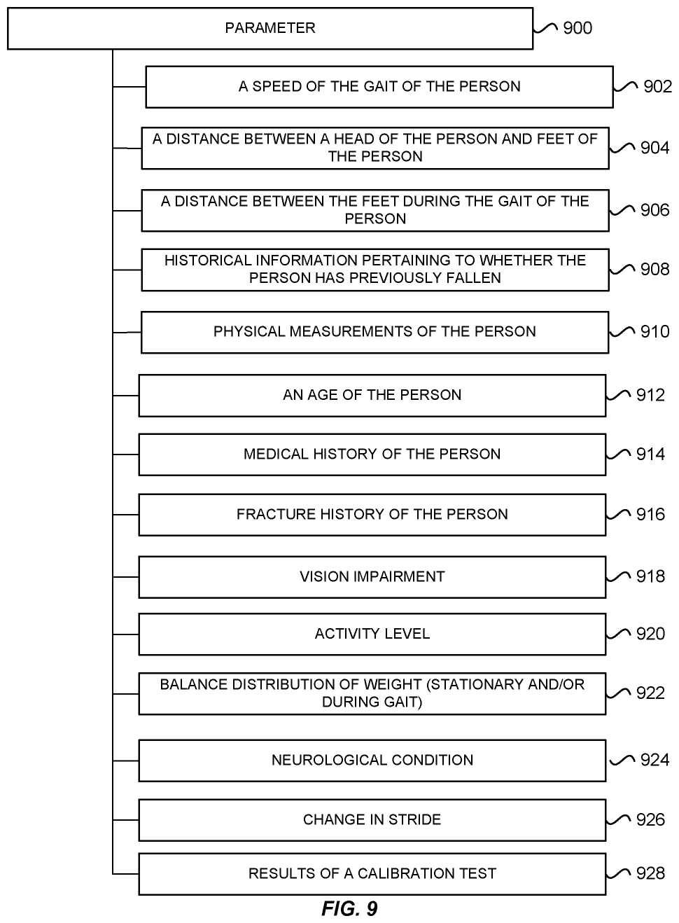

In some embodiments, the training data may include inputs of parameters (e.g., described below with regards to FIG. 9), variations in the parameters, variations in the parameters within a threshold time period, or some combination thereof and correlated outputs of an amount of gait deterioration for the parameters. That is, in some embodiments, there may be a separate respective machine learning model 154 for each individual parameter that is monitored. The respective machine learning model 154 may output the amount of gait deterioration for its particular parameter. The amount of gait deterioration may be a category (e.g., 1-5), a score (e.g., 1-5), a percentage (0-100%), or any suitable indicator of an amount of gait deterioration. The machine learning models 154 representing the various parameters may output the amount of gait deterioration, which is input into a result machine learning model 154 that determines the propensity for the fall event based on the amounts of gait deterioration or the amounts of gait deterioration within a threshold time period. The result machine learning model 154 may also determine the type of intervention(s) to perform based on the propensity for the fall event. In some embodiments, a single machine learning model may be used to monitor the parameter pertaining to the gait of the person based on the data, determine the amount of gait deterioration based on the parameter, and determine whether the propensity for the fall event for the person satisfies the threshold propensity condition.

The machine learning models 154 may be trained with the training data to perform an intervention based on the determined propensity for the fall event for the person. The propensity for the fall event may be represented by a category (e.g., 1-5), a score (e.g., 1-5), and/or a percentage (e.g., 0-100%). For example, if the propensity for the fall event is high (e.g., a 5), then a major intervention may be performed, such as contacting the computing device 15 of the medical personnel 27 caring for the person 25 to indicate that a fall event may occur soon. If the propensity for the fall event satisfies a threshold condition but is low (e.g., less than a 3), then a minor intervention may be performed, such as changing a property of the electronic device 13 (e.g., changing the color of light emitted).

In some embodiments, the cloud-based computing system 116 may include a database 129. The database 129 may store data pertaining to observations determined by the machine learning models 154. The observations may pertain to the amounts of gait deterioration for each parameter and/or the propensity for the fall event for the person 25. The observations may be stored by the database 129 over time to track the degradation and/or improvement of the parameters and/or the propensity for the fall event. Further, the observations may include indications of which types of interventions are successful in preventing the fall event or lessening the impact of a fall event. In some embodiments, the data received from the smart floor tile 112, moulding section 102, and/or the camera 50 may be correlated with an identity of the person 25 and/or the medical personnel 27 and stored in the database 129. The training data used to train the machine learning models 154 may be stored in the database 129.

The camera 50 may be any suitable camera capable of obtaining data including video and/or images and transmitting the video and/or images to the cloud-based computing system 116 via the network 20. The data obtained by the camera 50 may include timestamps for the video and/or images. In some embodiments, the cloud-based computing system 116 may perform computer vision to extract high-dimensional digital data from the data received from the camera 50 and produce numerical or symbolic information. The numerical or symbolic information may represent the parameters monitored pertaining to the gait of the person 25 monitored by the cloud-based computing system 116.

As described further below, gait baseline parameters may be calibrated prior to the cloud-based computing system 116 determines whether the propensity for the fall event satisfies the threshold propensity condition. One or more tests may be performed to calibrate the gait baseline parameters. For example, a smart floor tile test may involve the person 5 walking across the first room 21 while the smart floor tiles 112 measure pressure of the person's footsteps and transmit data representing the measured data (e.g., amount of pressure, location of pressure, timestamp of measurement, etc.) to the cloud-based computing system 116. The cloud-based computing system may calibrate gait baseline parameters for the gait speed of the person 25, width between feet during gait of the person 25, stride length of the person 25, and the like. The gait baseline parameters may be subsequently used to compare with subsequent data pertaining to the gait of the person 25 to determine the amount of gait deterioration and/or the propensity for a fall event of the person 25.

As depicted in FIG. 1A, a fall event (represented by dashed user 25) may be predicted by the cloud-based computing system 116 based on the data received from the smart floor tile 112, moulding sections 102, and/or the camera 50. The cloud-based computing system 116 may select and perform various interventions to prevent the fall event.

FIGS. 1C-1E depict various example configurations of smart floor tiles 112, and/or moulding sections 102 according to certain embodiments of this disclosure. FIG. 1C depicts an example system 10 that is used in a physical space of a smart building (e.g., care facility). The depicted physical space includes a wall 104, a ceiling 106, and a floor 108 that define a room. Numerous moulding sections 102A, 102B, 102C, and 102D are disposed in the physical space. For example, moulding sections 102A and 102B may form a baseboard or shoe moulding that is secured to the wall 108 and/or the floor 108. Moulding sections 102C and 102D may for a crown moulding that is secured to the wall 108 and/or the ceiling 106. Each moulding section 102A may have different shapes and/or sizes.

The moulding sections 102 may each include various components, such as electrical conductors, sensors, processors, memories, network interfaces, and so forth. The electrical conductors may be partially or wholly enclosed within one or more of the moulding sections. For example, one electrical conductor may be a communication cable that is partially enclosed within the moulding section and exposed externally to the moulding section to electrically couple with another electrical conductor in the wall 108. In some embodiments, the electrical conductor may be communicably connected to at least one smart floor tile 112. In some embodiments, the electrical conductor may be in electrical communication with a power supply 114. In some embodiments, the power supply 114 may provide electrical power that is in the form of mains electricity general-purpose alternating current. In some embodiments, the power supply 114 may be a battery, a generator, or the like.

In some embodiments, the electrical conductor is configured for wired data transmission. To that end, in some embodiments the electrical conductor may be communicably coupled via cable 118 to a central communication device 120 (e.g., a hub, a modem, a router, etc.). Central communication device 120 may create a network, such as a wide area network, a local area network, or the like. Other electronic devices 13 may be in wired and/or wireless communication with the central communication device 120. Accordingly, the moulding section 102 may transmit data to the central communication device 120 to transmit to the electronic devices 13. The data may be control instructions that cause, for example, an the electronic device 13 to change a property based on a prediction that the person 25 is going to experience a fall event. In some embodiments, the moulding section 102A may be in wired and/or wireless communication connection with the electronic device 13 without the use of the central communication device 120 via a network interface and/or cable. The electronic device 13 may be any suitable electronic device capable of changing an operational parameter in response to a control instruction.

In some embodiments, the electrical conductor may include an insulated electrical wiring assembly. In some embodiments, the electrical conductor may include a communications cable assembly. The moulding sections 102 may include a flame-retardant backing layer. The moulding sections 102 may be constructed using one or more materials selected from: wood, vinyl, rubber, fiberboard, and wood composite materials.

The moulding sections may be connected via one or more moulding connectors 110. A moulding connector 110 may enhance electrical conductivity between two moulding sections 102 by maintaining the conductivity between the electrical conductors of the two moulding sections 102. For example, the moulding connector 110 may include contacts and its own electrical conductor that forms a closed circuit when the two moulding sections are connected with the moulding connector 110. In some embodiments, the moulding connectors 110 may include a fiber optic relay to enhance the transfer of data between the moulding sections 102. It should be appreciated that the moulding sections 102 are modular and may be cut into any desired size to fit the dimensions of a perimeter of a physical space. The various sized portions of the moulding sections 102 may be connected with the moulding connectors 110 to maintain conductivity.

Moulding sections 102 may utilize a variety of sensing technologies, such as proximity sensors, optical sensors, membrane switches, pressure sensors, and/or capacitive sensors, to identify instances of an object proximate or located near the sensors in the moulding sections and to obtain data pertaining to a gait of the person 25. Proximity sensors may emit an electromagnetic field or a beam of electromagnetic radiation (infrared, for instance), and identify changes in the field or return signal. The object being sensed may be any suitable object, such as a human, an animal, a robot, furniture, appliances, and the like. Sensing devices in the moulding section may generate moulding section sensor data indicative of gait characteristics of the person 25, location (presence) of the person 25, the timestamp associated with the location of the person 25, and so forth.

The moulding section sensor data may be used alone or in combination with tile impression data generated by the smart floor tiles 112 and/or image data generated by the camera 50 to perform predict fall events for the person 25 and perform appropriate interventions to prevent the fall event from occurring. For example, the moulding section sensor data may be used to determine a control instruction to generate and to transmit to an electric device 13 and/or the smart floor tile 102A. The control instruction may include changing an operational parameter of the electronic device 13 based on the moulding section sensor data indicating the person 25 is going to experience a fall event. The control instruction may include instructing the smart floor tile 112 to reset one or more components based on an indication in the moulding section sensor data that the one or more components is malfunctioning and/or producing faulty results. Further, the moulding sections 102 may include a directional indicator (e.g., light) that is emits different colors of light, intensities of light, patterns of light, etc. based on a fall event being predicted by the cloud-based computing system 116.

In some embodiments, the moulding section sensor data can be used to verify the impression tile data and/or image data of the camera 50 is accurate for predicting a fall event for the person 25. Such a technique may improve accuracy of the determination. Further, if the moulding section sensor data, the impression tile data, and/or the image data do not align (e.g., the moulding section sensor data does not indicate a fall event will occur and the impression tile data indicates a fall event will occur), then further analysis may be performed. For example, tests can be performed to determine if there are defective sensors at the corresponding smart floor tile 112 and/or the corresponding moulding section 102 that generated the data. Further, control actions may be performed such as resetting one or more components of the moulding section 102 and/or the smart floor tile 112. In some embodiments, preference to certain data may be made by the cloud-based computing system 116. For example, in one embodiment, preference for the impression tile data may be made over the moulding section sensor data and/or the image data, such that if the impression tile data differs from the moudling section sensor data and/or the image data, the impression tile data is used to predict the propensity for the fall event.

FIG. 1D illustrates another configuration of the moulding sections 102. In this example, the moulding sections 102E-102H surround a border of a smart window 155. The moulding sections 102 are connected via the moulding connector 110. As may be appreciated, the modular nature of the moulding sections 102 with the moulding connectors 110 enables forming a square around the window. Other shapes may be formed using the moulding sections 102 and the moulding connectors 110.

The moulding sections 102 may be electrically and/or communicably connected to the smart window 155 via electrical conductors and/or interfaces. The moulding sections 102 may provide power to the smart window 155, receive data from the smart window 155, and/or transmit data to the smart window 155. One example smart window includes the ability to change light properties using voltage that may be provided by the moulding sections 102. The moulding sections 102 may provide the voltage to control the amount of light let into a room based on predicting a propensity for a fall event. For example, if the moulding section sensor data, impression tile data, and/or image data indicates the person 25 has a high propensity for experiencing a fall event, the cloud-based computing system 116 may perform an intervention by causing the moulding sections 102 to instruct the smart window 155 to change a light property to allow light into the room. In some instances the cloud-based computing system 116 may communicate directly with the smart window 155 (e.g., electronic device 13).

In some embodiments, the moulding sections 102 may use sensors to detect when the smart window 155 is opened. The moulding sections 102 may determine whether the smart window 155 opening is performed at an expected time (e.g., when a home owner is at home) or at an unexpected time (e.g., when the home owner is away from home). The moulding sections 102, the camera 50, and/or the smart floor tile 112 may sense the occupancy patterns of certain objects (e.g., people) in the space in which the moulding sections 102 are disposed to determine a schedule of the objects. The schedule may be referenced when determining if an undesired opening (e.g., break-in event) occurs and the moulding sections 102 may be communicatively to an alarm system to trigger the alarm when the certain event occurs.

The schedule may also be referenced when determining a medical condition of the person 25. For example, if the schedule indicates that the person 25 went to the bathroom a certain number of times (e.g., 10) within a certain time period (e.g., 1 hour), the cloud-based computing system 116 may determine that the person has a urinary tract infection (UTI) and may perform an intervention, such as transmitting a message to the computing device 12 of the person 25. The message may indicate the potential UTI and recommend that the person 25 schedules an appointment with a medical personnel.

As depicted, at least moulding section 102F is electrically and/or communicably coupled to smart shades 160. Again, the cloud-based computing system 116 may cause the moulding section 102F to control the smart shades 160 to extend or retract to control the amount of light let into a room. In some embodiments, the cloud-based computing system 116 may communicate directly with the smart shades 160.

FIG. 1E illustrates another configuration of the moulding sections 102 and smart floor tiles 112. In this example, the moulding sections 102E-102H surround a majority of a border of a smart door 170. The moulding sections 102J, 102K, and 102L and/or the smart floor tile 112 may be electrically and/or communicably connected to the smart door 170 via electrical conductors and/or interfaces. The moulding sections 102 and/or smart floor tiles 112 may provide power to the smart door 170, receive data from the smart door 170, and/or transmit data to the smart door 170. In some embodiments, the moulding sections 102 and/or smart floor tiles 112 may control operation of the smart door 170. For example, if the moulding section sensor data and/or impression tile data indicates that no one is present in a house for a certain period of time, the moulding sections 102 and/or smart floor tiles 112 may determine a locked state of the smart door 170 and generate and transmit a control instruction to the smart door 170 to lock the smart door 170 if the smart door 170 is in an unlocked state.

In another example, the moulding section sensor data, impression tile data, and/or the image data may be used to generate gait profiles for people in a smart building (e.g., care facility). When a certain person is in the room near the smart door 170, the cloud-based computing device 116 may detect that person's presence based on the data received from the smart floor tiles, moulding sections 102, and/or camera 50. In some embodiments, if the person 25 is detected near the smart door 170, the cloud-based computing system 116 may determine whether the person 25 has a particular medical condition (e.g., alzheimers) and/or a flag is set that the person should not be allowed to leave the smart building. If the person is detected near the smart door 170 and the person 25 has the particular medical condition and/or the flag set, then the cloud-based computing system 116 may cause the moulding sections 102 and/or smart floor tiles 112 to control the smart door 170 to lock the smart door 170. In some embodiments, the cloud-based computing system 116 may communicate directly with the smart door 170 to cause the smart door 170 to lock.

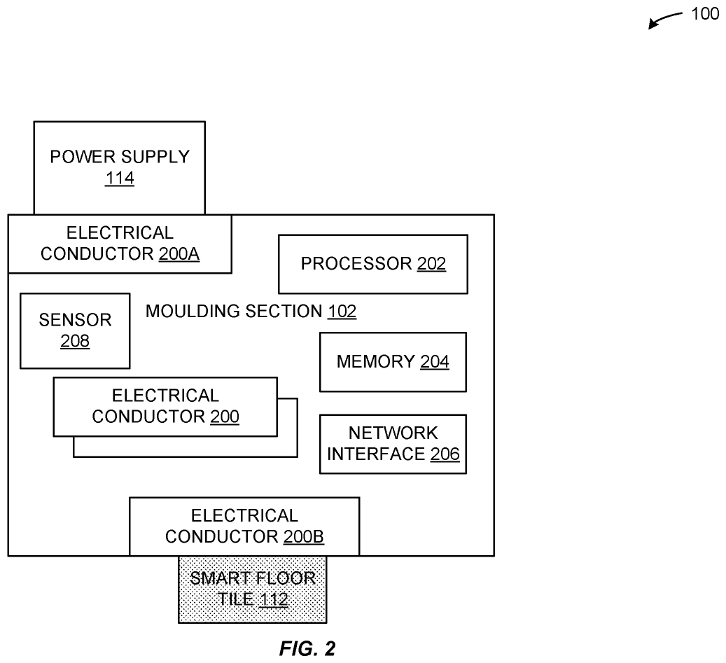

FIG. 2 illustrates an example component diagram of a moulding section 102 according to certain embodiments of this disclosure. As depicted, the moulding section 102 includes numerous electrical conductors 200, a processor 202, a memory 204, a network interface 206, and a sensor 208. More or fewer components may be included in the moulding section 102. The electrical conductors may be insulated electrical wiring assemblies, communications cable assemblies, power supply assemblies, and so forth. As depicted, one electrical conductor 200A may be in electrical communication with the power supply 114, and another electrical conductor 200B may be communicably connected to at least one smart floor tile 112.

In various embodiments, the moulding section 102 further comprises a processor 202. In the non-limiting example shown in FIG. 2, processor 202 is a low-energy microcontroller, such as the ATMEGA328P by Atmel Corporation. According to other embodiments, processor 202 is the processor provided in other processing platforms, such as the processors provided by tablets, notebook or server computers.

In the non-limiting example shown in FIG. 2, the moulding section 102 includes a memory 204. According to certain embodiments, memory 204 is a non-transitory memory containing program code to implement, for example, generation and transmission of control instructions, networking functionality, the algorithms for generating and analyzing locations, presence, and/or tracks, and the algorithms for determining gait deterioration and/or propensity for a fall event as described herein.

Additionally, according to certain embodiments, the moulding section 102 includes the network interface 206, which supports communication between the moulding section 102 and other devices in a network context in which smart building control using directional occupancy sensing and fall prediction/prevention is being implemented according to embodiments of this disclosure. In the non-limiting example shown in FIG. 2, network interface 206 includes circuitry 635 for sending and receiving data using Wi-Fi, including, without limitation at 900 MHz, 2.8 GHz and 5.0 GHz. Additionally, network interface 206 includes circuitry, such as Ethernet circuitry 640 for sending and receiving data (for example, smart floor tile data) over a wired connection. In some embodiments, network interface 206 further comprises circuitry for sending and receiving data using other wired or wireless communication protocols, such as Bluetooth Low Energy or Zigbee circuitry. The network interface 206 may enable communicating with the cloud-based computing device 116 via the network 20.

Additionally, according to certain embodiments, network interface 206 which operates to interconnect the moulding device 102 with one or more networks. Network interface 206 may, depending on embodiments, have a network address expressed as a node ID, a port number or an IP address. According to certain embodiments, network interface 206 is implemented as hardware, such as by a network interface card (NIC). Alternatively, network interface 206 may be implemented as software, such as by an instance of the java.net.NetworkInterface class. Additionally, according to some embodiments, network interface 206 supports communications over multiple protocols, such as TCP/IP as well as wireless protocols, such as 3G or Bluetooth. Network interface 206 may be in communication with the central communication device 120 in FIG. 1.

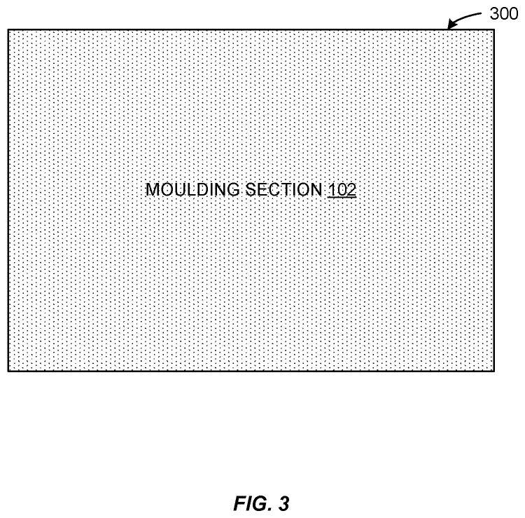

FIG. 3 illustrates an example backside view 300 of a moulding section 102 according to certain embodiments of this disclosure. As depicted by the dots 300, the backside of the moulding section 102 may include a fire-retardant backing layer positioned between the moulding section 102 and the wall to which the moulding section 102 is secured.

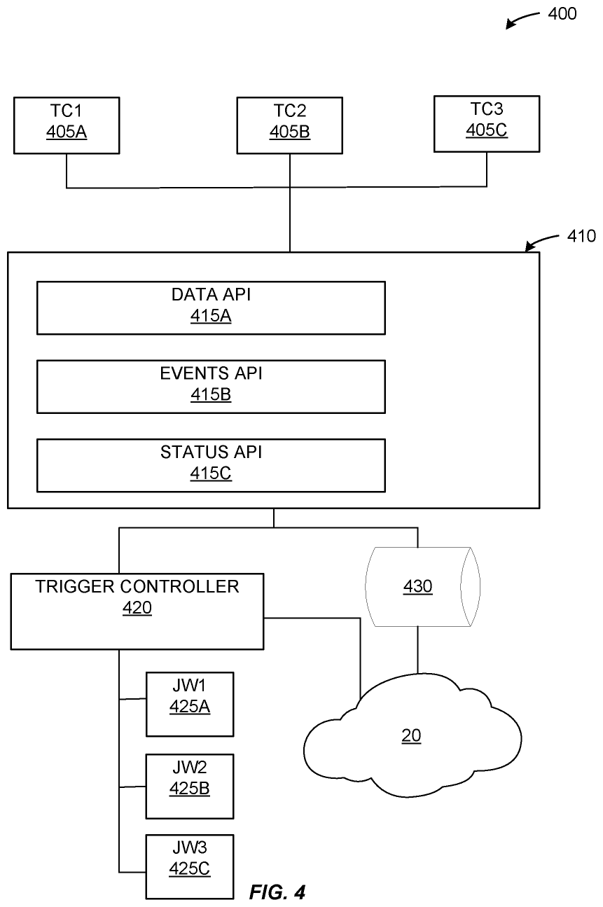

FIG. 4 illustrates a network and processing context 400 for smart building control using directional occupancy sensing and fall prediction/prevention according to certain embodiments of this disclosure. The embodiment of the network context 400 shown in FIG. 4 is for illustration only and other embodiments could be used without departing from the scope of the present disclosure.

In the non-limiting example shown in FIG. 4, a network context 400 includes one or more tile controllers 405A, 405B and 405C, an API suite 410, a trigger controller 420, job workers 425A-425C, a database 430 and a network 435.

According to certain embodiments, each of tile controllers 405A-405C is connected to a smart floor tile 112 in a physical space. Tile controllers 405A-405C generate floor contact data (also referred to as impression tile data herein) from smart floor tiles in a physical space and transmit the generated floor contact data to API suite 410. In some embodiments, data from tile controllers 405A-405C is provided to API suite 410 as a continuous stream. In the non-limiting example shown in FIG. 4, tile controllers 405A-405C provide the generated floor contact data from the smart floor tile to API suite 410 via the internet. Other embodiments, wherein tile controllers 405A-405C employ other mechanisms, such as a bus or Ethernet connection to provide the generated floor data to API suite 410 are possible and within the intended scope of this disclosure.

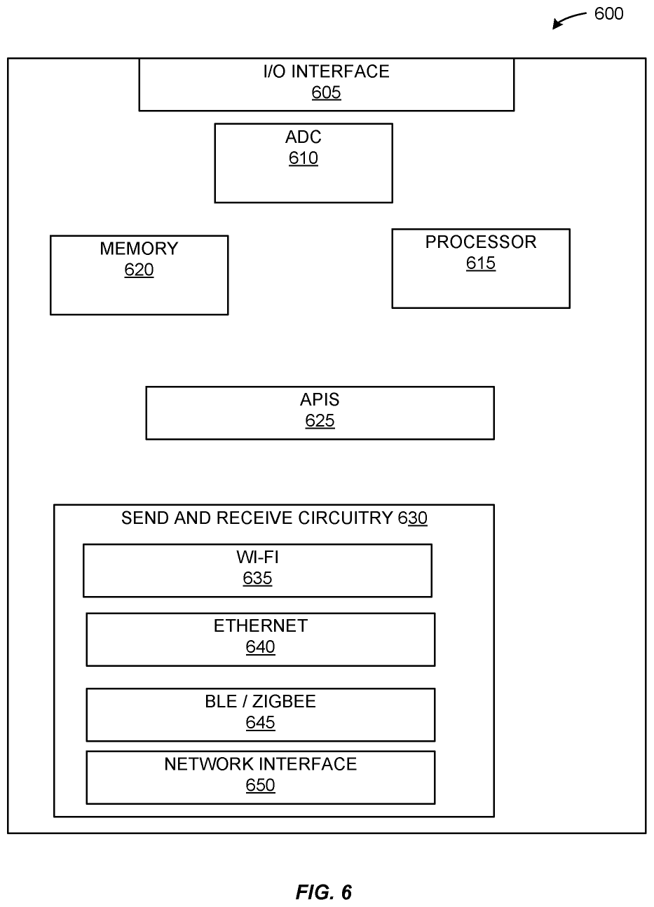

According to some embodiments, API suite 410 is embodied on a server 128 in the cloud-based computing system 116 connected via the internet to each of tile controllers 405A-405C. According to some embodiments, API suite is embodied on a master control device, such as master control device 600 shown in FIG. 6 of this disclosure. In the non-limiting example shown in FIG. 4, API suite 410 comprises a Data Application Programming Interface (API) 415A, an Events API 415B and a Status API 215C.

In some embodiments, Data API 415A is an API for receiving and recording tile data from each of tile controllers 405A-405C. Tile events include, for example, raw, or minimally processed data from the tile controllers, such as the time and data a particular smart floor tile was pressed and the duration of the period during which the smart floor tile was pressed. According to certain embodiments, Data API 415A stores the received tile events in a database such as database 430. In the non-limiting example shown in FIG. 4, some or all of the tile events are received by API suite 410 as a stream of event data from tile controllers 405A-405C, Data API 415A operates in conjunction with trigger controller 420 to generate and pass along triggers breaking the stream of tile event data into discrete portions for further analysis.

According to various embodiments, Events API 415B receives data from tile controllers 405A-405C and generates lower-level records of instantaneous contacts where a sensor of the smart floor tile is pressed and released.

In the non-limiting example shown in FIG. 4, Status API 415C receives data from each of tile controllers 405A-405C and generates records of the operational health (for example, CPU and memory usage, processor temperature, whether all of the sensors from which a tile controller receives inputs is operational) of each of tile controllers 405A-405C. According to certain embodiment, status API 415C stores the generated records of the tile controllers' operational health in database 430.

According to some embodiments, trigger controller 420 operates to orchestrate the processing and analysis of data received from tile controllers 405A-405C. In addition to working with data API 415A to define and set boundaries in the data stream from tile controllers 405A-405C to break the received data stream into tractably sized and logically defined “chunks” for processing, trigger controller 420 also sends triggers to job workers 425A-425C to perform processing and analysis tasks. The triggers comprise identifiers uniquely identifying each data processing job to be assigned to a job worker. In the non-limiting example shown in FIG. 4, the identifiers comprise: 1.) a sensor identifier (or an identifier otherwise uniquely identifying the location of contact); 2.) a time boundary start identifying a time in which the smart floor tile went from an idle state (for example, an completely open circuit, or, in the case of certain resistive sensors, a baseline or quiescent current level) to an active state (a closed circuit, or a current greater than the baseline or quiescent level); and 3.) a time boundary end defining the time in which a smart floor tile returned to the idle state.

In some embodiments, each of job workers 425A-425C corresponds to an instance of a process performed at a computing platform, (for example, cloud-based computing system 116 in FIG. 1) for determining tracks and performing an analysis of the tracks (e.g., such as predicting a propensity for a fall event and performing an intervention based on the propensity). Instances of processes may be added or subtracted depending on the number of events or possible events received by API suite 410 as part of the data stream from tile controllers 405A-205C. According to certain embodiments, job workers 425A-425C perform an analysis of the data received from tile controllers 405A-405C, the analysis having, in some embodiments, two stages. A first stage comprises deriving footsteps, and paths, or tracks, from impression tile data. A second stage comprises characterizing those footsteps, and paths, or tracks, to determine gait characteristics of the person 25. The gait characteristics may be presented to an online dashboard (in some embodiments, provided by a UI on an electronic device, such as computing device 12 or 15 in FIG. 1) and to generate control signals for devices (e.g., the computing devices 12 and/or 15, the electronic device 15, the moulding sections 102, the camera 50, and/or the smart floor tile 112 in FIG. 1) controlling operational parameters of a physical space where the smart floor impression tile data were recorded.

In the non-limiting example shown in FIG. 4, job workers 425A-425C perform the constituent processes of a method for analyzing smart floor tile impression tile data and/or moulding section sensor data to generate paths, or tracks. In some embodiments, an identity of the the person 25 may be correlated with the paths or tracks. For example, if the person scanned an ID badge when entering the physical space, their path may be recorded when the person takes their first step on a smart floor tile and their path may be correlated with an identifier received from scanning the badge. In this way, the paths of various people may be recorded (e.g., in a convention hall). This may be beneficial if certain people have desirable job titles (e.g., chief executive officer (CEO), vice president, president, etc.) and/or work at desirable client entities. For example, in some embodiments, the path of a CEO may be tracked in during a convention to determine which booths the CEO stopped at and/or an amount of time the CEO spent at each booth. Such data may be used to determine where to place certain booths in the future. For example, if a booth was visited by a threshold number of people having a certain title for a certain period of time, a recommendation may be generated and presented that recommends relocating the booth to a location in the convention hall that is more easily accessible to foot traffic. Likewise, if it is determined that a booth has poor visitation frequency based on the paths, or tracks, of attendees at the convention, a recommendation may be generated to relocate the booth to another location that is more easily accessible to foot traffic. In some embodiments, the machine learning models 154 may be trained to determine the paths, or tracks, of the people having various job titles and working for desired client entities, analyze their paths (e.g., which location the people visited, how long the people visited those locations, etc.), and generate recommendations.

According to certain embodiments, the method comprises the operations of obtaining impression image data, impression tile data, and/or moulding section sensor data from database 430, cleaning the obtained image data, impression tile data, and/or moulding section sensor data and reconstructing paths using the cleaned data. In some embodiments, cleaning the data includes removing extraneous sensor data, removing gaps between image data, impression tile data, and/or moulding section sensor data caused by sensor noise, removing long image data, impression tile data, and/or moulding section sensor data caused by objects placed on smart floor tiles, by objects placed in front of moulding sections, by objects stationary in image data, by defective sensors, and sorting image data, impression tile data, and/or moulding section sensor data by start time to produce sorted image data, impression tile data, and/or moulding section sensor data. According to certain embodiments, job workers 425A-425C perform processes for reconstructing paths by implementing algorithms that first cluster image data, impression tile data, and/or moulding section sensor data that overlap in time or are spatially adjacent. Next, the clustered data is searched, and pairs of image data, impression tile data, and/or moulding section sensor data that start or end within a few milliseconds of one another are combined into footsteps and/or locations of the object, which are then linked together to form footsteps and/or locations. Footsteps and/or locations are further analyzed and linked to create paths.

According to certain embodiments, database 430 provides a repository of raw and processed image data, smart floor tile impression tile data, and/or moulding section sensor data, as well as data relating to the health and status of each of tile controllers 405A-405C and moulding sections 102. In the non-limiting example shown in FIG. 4, database 430 is embodied on a server machine communicatively connected to the computing platforms providing API suite 410, trigger controller 420, and upon which job workers 425A-425C execute. According to some embodiments, database 430 is embodied on the cloud-based computing system 116 as the database 129.

In the non-limiting example shown in FIG. 4, the computing platforms providing trigger controller 420 and database 430 are communicatively connected to one or more network(s) 20. According to embodiments, network 20 comprises any network suitable for distributing impression tile data, image data, moulding section sensor data, determined paths, determined gait deterioration of a parameter, determine propensity for a fall event, and control signals (e.g., interventions) based on determined propensities for fall events, including, without limitation, the internet or a local network (for example, an intranet) of a smart building.

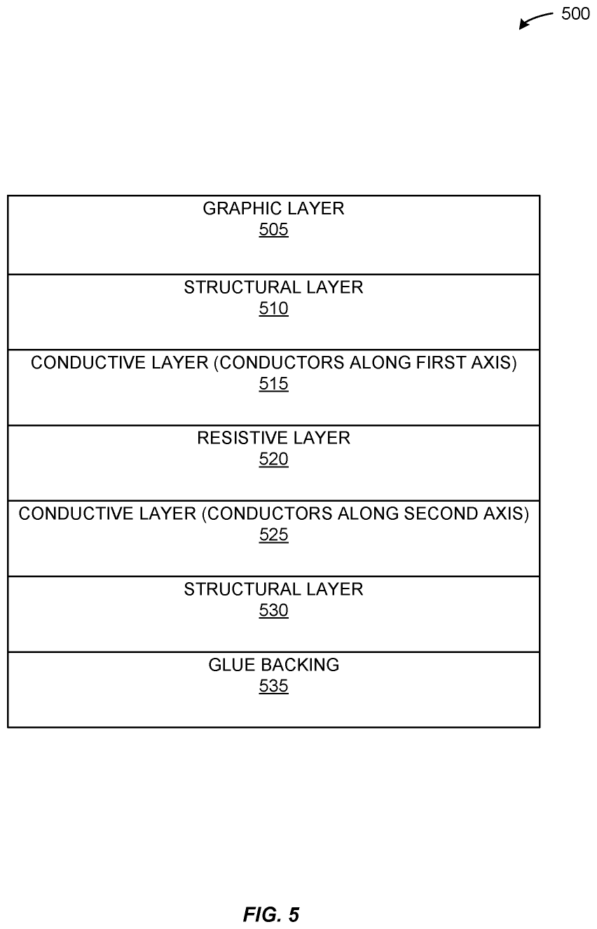

Smart floor tiles utilizing a variety of sensing technologies, such as membrane switches, pressure sensors and capacitive sensors, to identify instances of contact with a floor are within the contemplated scope of this disclosure. FIG. 5 illustrates aspects of a resistive smart floor tile 500 according to certain embodiments of the present disclosure. The embodiment of the resistive smart floor tile 500 shown in FIG. 5 is for illustration only and other embodiments could be used without departing from the scope of the present disclosure.

In the non-limiting example shown in FIG. 5, a cross section showing the layers of a resistive smart floor tile 500 is provided. According to some embodiments, the resistance to the passage of electrical current through the smart floor tile varies in response to contact pressure. From these changes in resistance, values corresponding to the pressure and location of the contact may be determined. In some embodiments, resistive smart floor tile 500 may comprise a modified carpet or vinyl floor tile, and have dimensions of approximately 2′×2′.

According to certain embodiments, resistive smart floor tile 500 is installed directly on a floor, with graphic layer 505 comprising the top-most layer relative to the floor. In some embodiments, graphic layer 505 comprises a layer of artwork applied to smart floor tile 500 prior to installation. Graphic layer 505 can variously be applied by screen printing or as a thermal film.

According to certain embodiments, a first structural layer 510 is disposed, or located, below graphic layer 505 and comprises one or more layers of durable material capable of flexing at least a few thousandths of an inch in response to footsteps or other sources of contact pressure. In some embodiments, first structural layer 510 may be made of carpet, vinyl or laminate material.

According to some embodiments, first conductive layer 515 is disposed, or located, below structural layer 510. According to some embodiments, first conductive layer 515 includes conductive traces or wires oriented along a first axis of a coordinate system. The conductive traces or wires of first conductive layer 515 are, in some embodiments, copper or silver conductive ink wires screen printed onto either first structural layer 510 or resistive layer 520. In other embodiments, the conductive traces or wires of first conductive layer 515 are metal foil tape or conductive thread embedded in structural layer 510. In the non-limiting example shown in FIG. 5, the wires or traces included in first conductive layer 515 are capable of being energized at low voltages on the order of 5 volts. In the non-limiting example shown in FIG. 5, connection points to a first sensor layer of another smart floor tile or to tile controller are provided at the edge of each smart floor tile 500.

In various embodiments, a resistive layer 520 is disposed, or located, below conductive layer 515. Resistive layer 520 comprises a thin layer of resistive material whose resistive properties change under pressure. For example, resistive layer 320 may be formed using a carbon-impregnated polyethylete film.

In the non-limiting example shown in FIG. 5, a second conductive layer 525 is disposed, or located, below resistive layer 520. According to certain embodiments, second conductive layer 525 is constructed similarly to first conductive layer 515, except that the wires or conductive traces of second conductive layer 525 are oriented along a second axis, such that when smart floor tile 500 is viewed from above, there are one or more points of intersection between the wires of first conductive layer 515 and second conductive layer 525. According to some embodiments, pressure applied to smart floor tile 500 completes an electrical circuit between a sensor box (for example, tile controller 425 as shown in FIG. 4) and smart floor tile, allowing a pressure-dependent current to flow through resistive layer 520 at a point of intersection between the wires of first conductive layer 515 and second conductive layer 525. The pressure-dependent current may represent a measurement of pressure and the measurement of pressure may be transmitted to the cloud-based computing system 116.

In some embodiments, a second structural layer 530 resides beneath second conductive layer 525. In the non-limiting example shown in FIG. 5, second structural layer 530 comprises a layer of rubber or a similar material to keep smart floor tile 500 from sliding during installation and to provide a stable substrate to which an adhesive, such as glue backing layer 535 can be applied without interference to the wires of second conductive layer 525.

The foregoing description is purely descriptive and variations thereon are contemplated as being within the intended scope of this disclosure. For example, in some embodiments, smart floor tiles according to this disclosure may omit certain layers, such as glue backing layer 535 and graphic layer 505 described in the non-limiting example shown in FIG. 5.

According to some embodiments, a glue backing layer 535 comprises the bottom-most layer of smart floor tile 500. In the non-limiting example shown in FIG. 5, glue backing layer 535 comprises a film of a floor tile glue.

FIG. 6 illustrates a master control device 600 according to certain embodiments of this disclosure. FIG. 6 illustrates a master control device 600 according to certain embodiments of this disclosure. The embodiment of the master control device 600 shown in FIG. 6 is for illustration only and other embodiments could be used without departing from the scope of the present disclosure.

In the non-limiting example shown in FIG. 6, master control device 600 is embodied on a standalone computing platform connected, via a network, to a series of end devices (e.g., tile controller 405A in FIG. 4) in other embodiments, master control device 600 connects directly to, and receives raw signals from, one or more smart floor tiles (for example, smart floor tile 500 in FIG. 5). In some embodiments, the master control device 600 is implemented on a server 128 of the cloud-based computing system 116 in FIG. 1B and communicates with the smart floor tiles 112, the moulding sections 102, the camera 50, the computing device 12, the computing device 15, and/or the electronic device 13.

According to certain embodiments, master control device 600 includes one or more input/output interfaces (I/O) 605. In the non-limiting example shown in FIG. 6, I/O interface 605 provides terminals that connect to each of the various conductive traces of the smart floor tiles deployed in a physical space. Further, in systems where membrane switches or smart floor tiles are used as mat presence sensors, I/O interface 605 electrifies certain traces (for example, the traces contained in a first conductive layer, such as conductive layer 515 in FIG. 5) and provides a ground or reference value for certain other traces (for example, the traces contained in a second conductive layer, such as conductive layer 525 in FIG. 5). Additionally, I/O interface 605 also measures current flows or voltage drops associated with occupant presence events, such as a person's foot squashing a membrane switch to complete a circuit, or compressing a resistive smart floor tile, causing a change in a current flow across certain traces. In some embodiments, I/O interface 605 amplifies or performs an analog cleanup (such as high or low pass filtering) of the raw signals from the smart floor tiles in the physical space in preparation for further processing.

In some embodiments, master control device 600 includes an analog-to-digital converter (“ADC”) 610. In embodiments where the smart floor tiles in the physical space output an analog signal (such as in the case of resistive smart floor tile), ADC 610 digitizes the analog signals. Further, in some embodiments, ADC 610 augments the converted signal with metadata identifying, for example, the trace(s) from which the converted signal was received, and time data associated with the signal. In this way, the various signals from smart floor tiles can be associated with touch events occurring in a coordinate system for the physical space at defined times. While in the non-limiting example shown in FIG. 6, ADC 610 is shown as a separate component of master control device 600, the present disclosure is not so limiting, and embodiments wherein ADC 610 is part of, for example, I/O interface 605 or processor 615 are contemplated as being within the scope of this disclosure.

In various embodiments, master control device 600 further comprises a processor 615. In the non-limiting example shown in FIG. 6, processor 615 is a low-energy microcontroller, such as the ATMEGA328P by Atmel Corporation. According to other embodiments, processor 615 is the processor provided in other processing platforms, such as the processors provided by tablets, notebook or server computers.

In the non-limiting example shown in FIG. 6, master control device 600 includes a memory 620. According to certain embodiments, memory 620 is a non-transitory memory containing program code to implement, for example, APIs 625, networking functionality and the algorithms for generating and analyzing tracks and predicting/preventing fall events by performing interventions described herein.

Additionally, according to certain embodiments, master control device 600 includes one or more Application Programming Interfaces (APIs) 625. In the non-limiting example shown in FIG. 6, APIs 625 include APIs for determining and assigning break points in one or more streams of smart floor tile data and/or moulding section sensor data and defining data sets for further processing. Additionally, in the non-limiting example shown in FIG. 6, APIs 625 include APIs for interfacing with a job scheduler (for example, trigger controller 420 in FIG. 4) for assigning batches of data to processes for analysis and determination of tracks and predicting/preventing fall events using interventions. According to some embodiments, APIs 625 include APIs for interfacing with one or more reporting or control applications provided on a client device. Still further, in some embodiments, APIs 625 include APIs for storing and retrieving image data, smart floor tile data, and/or moulding section sensor data in one or more remote data stores (for example, database 430 in FIG. 4, database 129 in FIG. 1B, etc.).

According to some embodiments, master control device 600 includes send and receive circuitry 630, which supports communication between master control device 600 and other devices in a network context in which smart building control using directional occupancy sensing is being implemented according to embodiments of this disclosure. In the non-limiting example shown in FIG. 6, send and receive circuitry 630 includes circuitry 635 for sending and receiving data using Wi-Fi, including, without limitation at 900 MHz, 2.8 GHz and 5.0 GHz. Additionally, send and receive circuitry 630 includes circuitry, such as Ethernet circuitry 640 for sending and receiving data (for example, smart floor tile data) over a wired connection. In some embodiments, send and receive circuitry 630 further comprises circuitry for sending and receiving data using other wired or wireless communication protocols, such as Bluetooth Low Energy or Zigbee circuitry.

Additionally, according to certain embodiments, send and receive circuitry 630 includes a network interface 650, which operates to interconnect master control device 600 with one or more networks. Network interface 650 may, depending on embodiments, have a network address expressed as a node ID, a port number or an IP address. According to certain embodiments, network interface 650 is implemented as hardware, such as by a network interface card (NIC). Alternatively, network interface 650 may be implemented as software, such as by an instance of the java.net.NetworkInterface class. Additionally, according to some embodiments, network interface 650 supports communications over multiple protocols, such as TCP/IP as well as wireless protocols, such as 3G or Bluetooth.

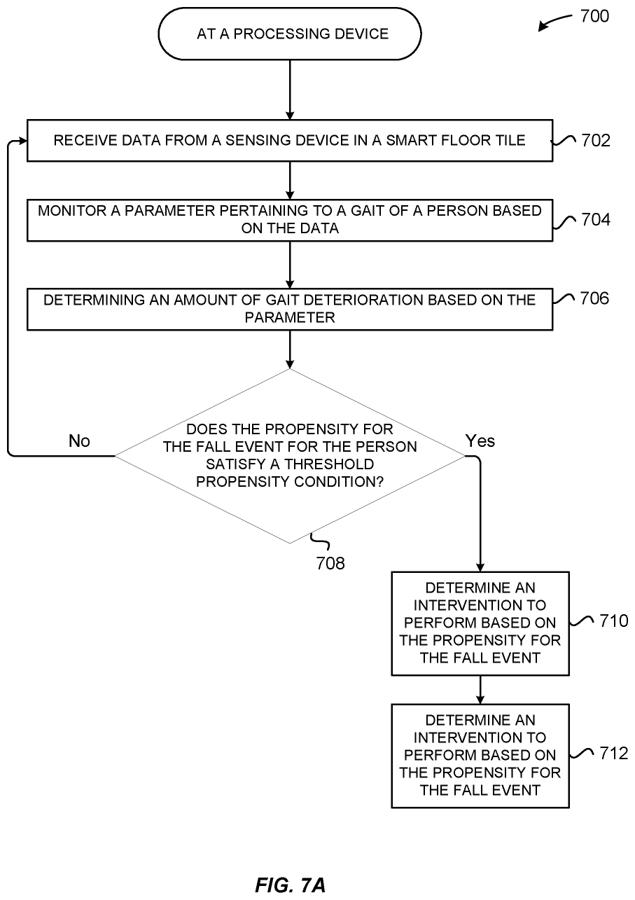

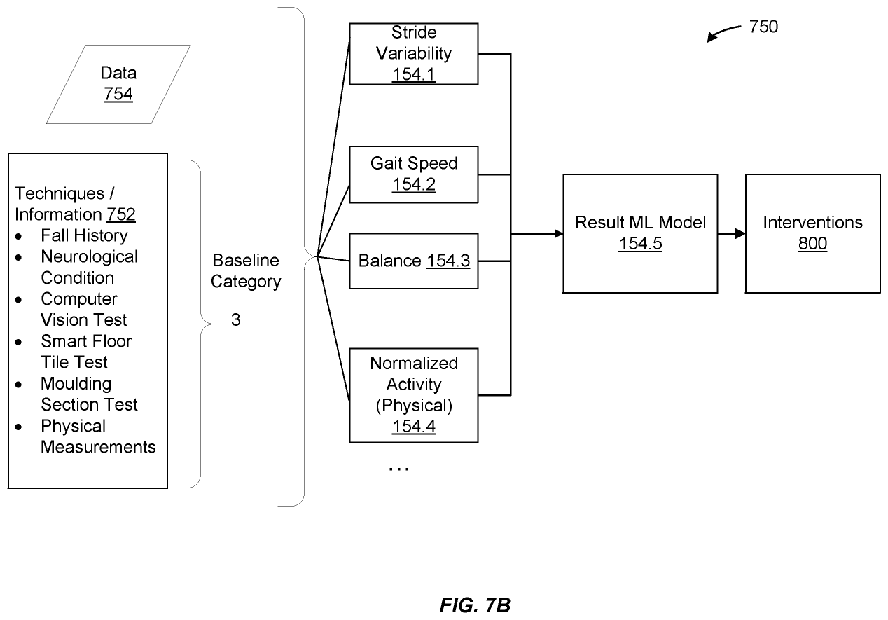

FIG. 7A illustrates an example of a method 700 for predicting a fall event according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 702, the processing device may receive data from a sensing device in a smart floor tile 112. The data may be pressure measured by a person stepping on the smart floor tile 112 with one or both of their feet. The data may include a specific coordinate where the pressure is measured (e.g., an identity of the sensing device that is pressed in the smart floor tile 112 may be included with the data and the location of that particular sensing device is stored in the database 129) by the sensing device, an amount of pressure applied to the sensing device, a time at which the pressure is applied to the sensing device, and so forth. In some embodiments, data may be received from the moulding section 102 and/or the camera 50. In embodiments where the parameter is monitored using the camera, the processing device may use computer vision, object recognition, measured pressure, location of feet of the person, or some combination thereof.

At block 704, the processing device may monitor a parameter pertaining to a gait of a person based on the data. The parameters are discussed in detail with regard to FIG. 9 below. Monitoring the parameter may include determining a category for the person based on the value of the parameter. The category may range from 1 to 5 where 1 is correlated with a least likely chance of the person falling and a 5 is correlated with a highest chance of the person falling. The person may be re-categorized while they are located in the physical space with the smart floor tiles 112, the moulding sections 102, and/or the camera 50. For example, the progression of the person from a category 1 to 5 for a propensity for a fall event to occur may be tracked and a time differential of how long it took for the person to move between categories may be determined and used to determine what intervention to perform. The categories for the propensity for the fall event may ebb and flow as the person improves and/or worsens a health condition.

At an initial time, as described below, the person may be categorized for one or more parameters and the categories may serve as one or more gait baseline parameters to use to compare against categories that are assigned to the person for the one or more parameters at a later time. The one or more gait baseline parameters may be stored as part of a motion profile for the person in the database 129 of the cloud-based computing system 116. The motion profile may include an average gait speed of the person, paths the person takes during a day and the times at which the person takes those paths, average width of feet from each other during gait, length of stride, and so forth.

However, in some instances, the person may not receive the one or more initial categories (gait baseline parameters). In such an embodiment, the processing device may use historical information pertaining to gait and/or balance that are characteristic of a propensity for a person to experience a fall event. The historical information may be obtained from a large group of people over a period of time and may be correlated with whether the people in the group experienced fall events. The historical information may be any combination of parameters including physical measurements (e.g., weight, height), personal statistics (e.g., age, gender, demographic information, etc.), medical history, neurological conditions, medications, fall history, gait characteristics (e.g., gait speed reduction within a certain time period, width of feet during gait, proximity of head to feet during gait, etc.), balance characteristics, and the like. For example, if the processing device determines the person has fallen in the past and the width of the person's feet are within a certain range, the processing device may determine the propensity for the person to experience a fall event warrants an intervention. Any suitable combination of historical information may be used to determine whether the person is likely to experience a fall event without using a gait baseline parameter.

At block 706, the processing device may determine an amount of gait deterioration based on the parameter. The amount of gait deterioration may be any suitable indication, such as a category (e.g., 1-5), a score (e.g., 1-5), a percentage (0-100%), and the like. In some embodiments, the amount of gait deterioration may be based on the category, score, or percentage for a particular parameter changing a certain amount within a certain time period. For example, the gait deterioration may be determined to be high if the category for a parameter changed from a 1 to a 5 within a short amount of time (e.g., minutes).

At block 708, the processing device may determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period. The propensity for a fall event may refer to a score (e.g., 1-5), a category (e.g., 1-5), percentage (e.g., 0-100%), or any suitable indication that is tied to how likely the person is to experiencing a fall event. The propensity for the fall event may be determined based on a category, score, or percentage for one parameter or any suitable combination of categories, scores, or percentages for parameters. For example, if the gait speed of the person deteriorated by 50% and the stride length of the person deteriorated by 50%, then the propensity for the fall event may be categorized at a high level (e.g., 4), and if the gait speed of the person deteriorated by 10% and the stride length of the person deteriorated by 5%, then the propensity for the fall event may be categorized a low level (e.g., 1).

In some embodiments, the threshold propensity condition may be satisfied when the amount of gait deterioration satisfies a threshold deterioration condition. For example, if the threshold deterioration condition specifies the amount of gait deterioration has to exceed a certain value (e.g., category of 3, score of 3, a percentage (50%), etc.) and the amount of gait deterioration exceeds the certain value, then the threshold propensity condition may be satisfied.

In some embodiments, the threshold propensity condition may be satisfied when the amount of gait deterioration satisfies a threshold deterioration condition within a threshold time period. For example, if the threshold deterioration condition specifies the amount of gait deterioration has to exceed a certain value (e.g., category of 3, score of 3, a percentage (50%), etc.) within the threshold time period (e.g., minutes, hours, days, etc.), and the amount of gait deterioration exceeds the certain value within that threshold time period (e.g., the amount of gait deterioration changed from 5% to 50% within an hour), then the threshold propensity condition may be satisfied.

If the propensity for the fall event for the person does not satisfy the threshold propensity condition, the processing device may return to block 702 to receive subsequent data from the sensing device in the smart floor tile 112 and continue to perform the other operations specified in the blocks 704, 706, and 708 until the propensity for the fall event for the person satisfies the threshold propensity condition.

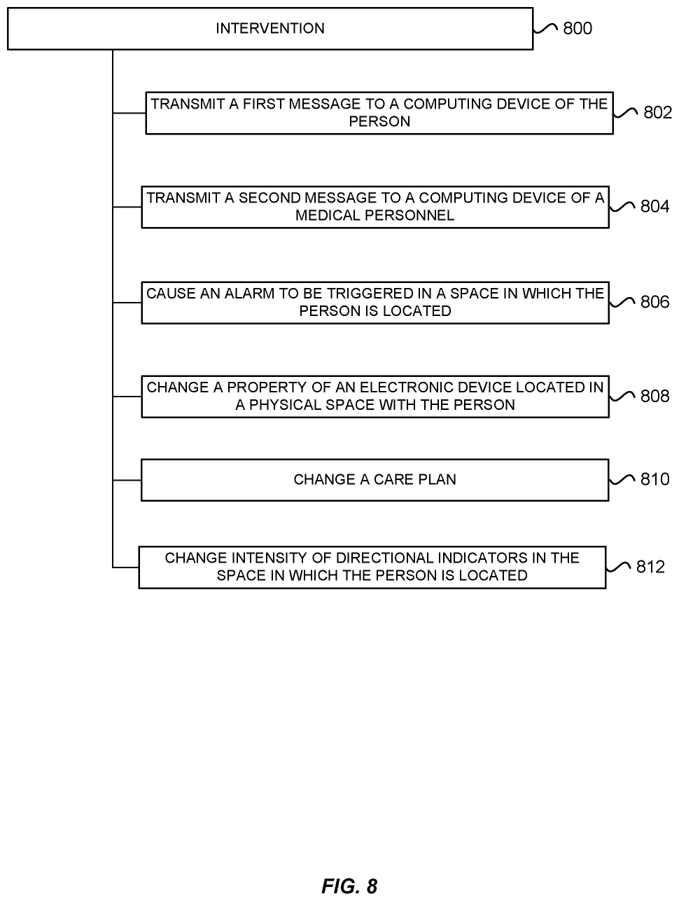

If the propensity for the fall event for the person satisfies the threshold propensity condition, then at block 710, the processing device determines an intervention to perform based on the propensity for the fall event. Various types of interventions are discussed in detail with regard to FIG. 8 below. There may be varying types of interventions with varying levels of severity that are associated with different levels of the propensity for the fall event. The interventions may escalate in severity based on how imminent the fall event is to occurring determined by the propensity for the fall event. Once one or more interventions are selected, the processing device may perform the one or more interventions.

In some embodiments, the monitoring the parameter pertaining to the gait of the person based on the data (block 704), the determining the amount of gait deterioration based on the parameter (block 706), and/or the determining whether the propensity for the fall event for the person satisfies the threshold propensity condition may include inputting the data into one or more machine learning models 154. The one or more machine learning models 154 may be trained to determine the amount of gait deterioration based on the parameter and to determine whether the propensity for the fall event for the person satisfies the threshold propensity condition.

In some embodiments, the effectiveness of the interventions that are performed may be tracked and a feedback loop may be used to update the one or more machine learning models 154. For example, the smart floor tiles 112, moulding sections 102, and/or camera 50 may obtain data that indicates whether the person fell or not after the intervention is performed. That data may be transmitted to the cloud-based computing system 116, which may update the machine learning models to either perform different interventions in the future if the intervention(s) performed did not work or continue to perform the same interventions if the interventions did work.

FIG. 7B illustrates an example architecture 750 including machine learning models 154 to perform the method of FIG. 7A according to certain embodiments of this disclosure. In some embodiments, each parameter that is monitored may be associated with a calibrated gait baseline parameter. The one or more gait baseline parameters may be combined using a function that weights the various gait baseline parameters to determine a baseline category, score, or percentage. Some embodiments may use certain information and/or techniques 752 when determining the one or more gait baseline parameters. Each of the gait baseline parameters may be stored in the database 129.

For example, the information and/or techniques 752 may include the fall history of the person. Research has shown that if a person has previously fallen, the person may be more likely to fall again in the future. The information and/or techniques 752 may include any neurological condition of the person. Certain neurological conditions may increase the likelihood that the person will fall. For example, if the person has epilepsy, the person may be prone to seizures that cause the person to fall while walking.

The information and/or techniques 752 may include a computer vision test. The camera 50 may stream video and/or images of the person during gait in a physical space (e.g., a care room). Using data received from the camera 50, the cloud-based computing system 116 may analyze the parameters of the person using computer vision to set the gait baseline parameters.

For example, computer vision may be used to determine an average gait stride length of the person, an average gait speed, an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

The information and/or techniques 752 may include a smart floor tile test. The smart floor tile test may involve receiving data from the smart floor tiles in the space in which the person is located while the person gaits. The data may include pressure measurements, location of pressure, time at which the pressure is measured, and so forth. The data may be used to determine an average gait stride length of the person, an average gait speed (e.g., differences in timestamps of detected footsteps from the smart floor tiles), an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

The information and/or techniques 752 may include moulding section testing. The moulding section test may involve receiving data from the moulding sections in the space in which the person is located while the person gaits. The data may include a silhouette of the person during the test as they gait in the space. The silhouette may be obtained using infrared imaging and/or proximity sensors that track the location of the person and the body parts of the person during the test as they gait. The data may be used to determine an average gait stride length of the person, an average gait speed (e.g., differences in timestamps of detected footsteps from the smart floor tiles), an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

In some embodiments, some combination of the computer vision test, the smart floor tile test, and/or the moulding section test may be used to calibrate the gait baseline parameters for the person.

The information and/or techniques 752 may include physical measurements of the person (e.g., height, weight, body weight distribution, body mass index, etc.) and other personal information about the person (e.g., age, medical history, gender, medications, and the like).

The one or more gait baseline parameters may be used in any combination to determine a baseline category for the propensity of the person to experience a fall event. In the depicted embodiment, the baseline category is determined to be a 3 in a range of 1-5 where 1 is the least likely to experience a fall event and a 5 is the most likely to experience a fall event. The one or more baseline parameters and/or the baseline category may be stored in the database 129.

The cloud-based computing system 116 may receive data 754 from the smart floor tiles 112, the moulding sections 102, and/or the camera 50. The data may be input into one or more machine learning models 154 that are each trained to monitor a particular parameter using the data and determine an amount of gait deterioration based on the monitored parameter. For example, the machine learning models 154 include a stride variability machine learning model 154.1, a walking speed machine learning model 154.2, a balance machine learning model 154.3, and a normalized activity (physical) machine learning mode 154.4. The machine learning models 154.1-154.4 may be trained to determine an amount of gait deterioration for a particular parameter. The amount of gait deterioration may include a category, a score, a rate, a percentage, or any suitable indicator the provides a measurement of the amount of gait deterioration.

The stride variability machine learning model 154.1 may be trained using training data that is labeled to indicate that stride variability, in terms of stride time (e.g., how long it takes a person to perform a stride during gait), stride length (e.g., a distance of a stride), or both, is correlated with a certain amount of gait deterioration. Further the stride variability machine learning model 154.1 may be trained to determine that the change in the characteristics of the stride occurring within certain periods of time is correlated with a certain amount of gait deterioration.

The gait speed machine learning model 154.2 may be trained using training data that is labeled to indicate that gait speed, in terms of how fast the person walks, is correlated with a certain amount of gait deterioration. Further the stride variability machine learning model 154.1 may be trained to determine that the change (e.g., reduction) in gait speed occurring within certain periods of time is correlated with a certain amount of gait deterioration.

The balance machine learning model 154.3 may be trained using training data that is labeled to indicate that the person is exhibiting a certain amount of balance is correlated with a certain amount of gait deterioration. The amount of balance may be measured in by body sway that may occur in any plane of motion. Sway may be determined based on analyzing the footsteps of the person and/or distribution of weight of the person as detected by the smart floor tiles 112, by analyzing body motion using video data from the camera 50 and/or data obtained from the moulding sections 102. Impaired balance may be used to predict the propensity for the fall event to occur. Further the stride variability machine learning model 154.1 may be trained to determine that the change in the balance of the person occurring within certain periods of time is correlated with a certain amount of gait deterioration.

The normalized activity machine learning model 154.2 may be trained using training data that is labeled to indicate that certain physical traits of a person are correlated with a certain amount of gait deterioration. For example, changes in the height, weight, age, weight distribution, body mass index, medical conditions, fall history, activity levels, and the like, may contribute to gait deterioration. Further the normalized activity machine learning model 154.1 may be trained to determine that the change in the physical traits occurring within certain periods of time is correlated with a certain amount of gait deterioration.

As depicted, any suitable number of machine learning models 154 (up to parameter machine learning model N) may be trained and used to determine the amount of gait deterioration as it pertains to a particular parameter. The output of the machine learning models 154.1 through 154.4 associated with the respective parameters may be input to a result machine learning model 154.5.

The result machine learning model 154.5 may be trained to analyze the various amounts of gait deterioration for the respective parameters represented by the respective machine learning models 154.1-154.4 and determine a propensity for the fall event. In some embodiments, the amount of gait deterioration for each parameter that is output by the machine learning models 154.1-154.4 may be compared with a respective corresponding gait baseline parameter when determining the propensity for the fall event. Each amount of gait deterioration may be considered a flag if the amount of gait deterioration satisfies a threshold deterioration condition. In some embodiments, the larger the number of flags that are present for the person, the higher the propensity for the fall event to occur for the person. That is, if there are flags present for the amount of gait deterioration determined by the stride variability machine learning model 154.1, the gait speed machine learning model 154.2, the balance machine learning model 154.3, and the normalized activity machine learning model 154.4, then the propensity for the fall event for the person may be high. In contrast, if there is just one flag present for the stride variability machine learning model 154.1, then the propensity for the fall event may be low.

In some embodiments, the propensity for the fall event may be compared with the baseline category to determine whether the propensity for the fall event satisfies the threshold propensity condition. For example, if the propensity for the fall event varies from the baseline category by a threshold amount (e.g., 1, 2, 3, etc.), then the propensity for the fall event may satisfy the threshold propensity condition.

Further, some machine learning models 154.1-154.4 may be associated with higher priority parameters and their output may be weighted differently when compared with the output of the other machine learning models corresponding to lesser priority parameters. For example, balance may be considered a high priority flag in indicating a fall event, and thus, the amount of gait deterioration determined for balance by the balance machine learning model 154.3 may be weighted more heavily that outputs of the other machine learning models 154.1, 154.2, and/or 154.4.

The result machine learning model 154.5 may also determine one or more interventions to perform based on the propensity for the fall event for the person. More severe interventions may be selected if the propensity for the fall event is high, and less severe interventions may be selected if the propensity for the fall event is low.

FIG. 8 illustrates example interventions 800 according to certain embodiments of this disclosure. The interventions 800 may each be associated with a level of severity. Less severe interventions 800 may be selected and performed for people having lower propensity for a fall event to occur, and more severe interventions 800 may be selected and performed for people having higher propensity for the fall event to occur. The interventions 800 are provided as examples and are not intended to limit the scope of the disclosure. Additional interventions 800 or fewer interventions 800 may be used in some embodiments.

A first intervention 802 may include transmitting a message to a computing device 12 of the person (e.g., elderly patient) for which the propensity of the fall event satisfies the threshold propensity condition. The message may include a notification that the fall event is likely to occur and/or instructs the user to stop walking, grab onto a supporting structure, change a gait speed, change the width of their feet, change their distribution of weight, and the like.

A second intervention 804 may include transmitting a message to a computing device of the medical personnel (e.g., nurse) that is on duty and/or assigned to care for the person. For example, the message may include a notification to the medical personnel that indicates the person is about to experience a fall event. The message may include a name of the person, which room the person is located, and/or a likelihood that the person is going to fall, among other things. For example, the message may include information about previous fall history for the person, known medical conditions of the person, fracture history of the person, age, medications taken by the person, and/or any suitable information that may aid the medical personnel in treating the person if the fall event occurs before the medical personnel arrives and/or if the medical personnel is able to prevent the fall. In some embodiments, the message may include a notification that reassigns the medical personnel to a station in closer proximity to or in farther proximity from the room where the person is located.

A third intervention 806 may causing an alarm to be triggered in a space in which the person is located. The alarm may be disposed at a nursing station that emits a certain audible, visual, and/or haptic indication that is represents the fall event may occur. The alarm may be disposed in the room in which the person is located and may emit a certain audible, visual, and/or haptic indication that is represents the fall event may occur.

A fourth intervention 808 may include changing a property of an electronic device located in a physical space with the person. For example, a smart light installed in the room in which the person is located may be controlled to emit a certain color of light and/or pattern of light, a smart thermostat may be controlled to change a temperature, a smart device located on the floor (e.g., smart vacuum) may be controlled to return to its home base to clear the way for the person to gait, a smart speaker may be controlled to play music and/or emit a warning about the fall event, and the like.

A fifth intervention 810 may include changing a care plan for the person. The care plan may be changed to instruct the person to complete a puzzle within a certain time period and/or perform any mentally stimulating activity that is correlated with improved mental capabilities. Improving mental capabilities may aid in reducing the likelihood of the person experiencing a fall event. The change in the care plan may relate to a diet of the person, different medication to prescribe to the person, an activity plan for the person, laboratory tests to perform for the person, medical examinations to perform for the person, and so forth.

A sixth intervention 812 may include changing an intensity of one or more directional indicators in the space in which the person is located. In some embodiments, the directional indicators may be lights, a display, audio speakers, and the like that are included in the moulding sections 102. In some embodiments, the directional indicators may be any suitable electronic device in the space in which the person is located that is capable of providing an indication of a direction for the person to move.

FIG. 9 illustrates example parameters 900 that may be monitored according to certain embodiments of this disclosure. Some of the parameters may have higher priority in terms of indicating whether a fall event may occur and those parameters may receive a higher weight when determining the propensity for the fall event. The parameters 900 are provided as examples and are not intended to limit the scope of the disclosure. Additional parameters 900 or fewer parameters 900 may be used in some embodiments.

A first parameter 902 may include a speed of the gait of the person. Gait speed may be determined based on the footsteps and how quickly the footsteps are made using the data from the smart floor tile 112, the moulding sections 102, and/or the camera 50. For example, the impression tile data received from the smart floor tile 112 may include the measured pressure associated with the footsteps and timestamps at which the pressure is measured. Such timestamps may be used to determine the speed at which the person is walking. Research has shown that reduced gait speed is an indicator of a propensity for a fall event.

A second parameter 904 may include a distance between a head of the person and feet of the person. Data received from the camera 50 and/or the moulding sections 102 may be used to determine the distance between the head of the person and feet of the person. Research has shown that the closer a person's head is to their feet, the more likely they are to fall because their center of gravity is off balance. As people age, their posture tends to decline and their heads often get closer to their feet as they hunch over. A reduction in distance between the head and feet of a person is an indicator of a propensity for a fall event.

A third parameter 906 may include a distance between the feet of the person during the gait of the person. The distance may be a width between the left and right foot. The distance may be a length of the stride between the left and right foot. If the width of the feet reduces, research has shown that is an indicator for a propensity for a fall event.

A fourth parameter 908 may include historical information pertaining to whether the person has previously fallen. Research shows that a person is more likely to fall again if that person has already experienced a fall event in the past.

A fifth parameter 910 may include physical measurements of the person. For example, the physical measurements may include height, weight, body mass index, weight distribution, and so forth. Certain physical measurements may be indicative of a propensity for a fall event to occur.

A sixth parameter 912 may include an age of the person. Research shows people over a certain age (e.g., 60) are more likely to experience a fall event because their muscles and skeletal strength weakens.

A seventh parameter 914 may include a medical history of the person. For example, if the person has a disease or medical condition, then that may indicate a propensity for a fall event.

An either parameter 916 may include a fracture history of the person. For example, if the person has previously fractured their hip, then that may indicate a propensity for a fall event.

A ninth parameter 918 may include vision impairment of the person. For example, if the person has poor eyesight, then that may indicate a propensity for a fall event (e.g., the person may not be able to see the floor is wet).

A tenth parameter 920 may include an activity level of the person. For example, if the person is rarely active, then their muscles may be atrophied. As a result, the person may be more likely to experience a fall event if they are not active.

An eleventh parameter 922 may include a balance distribution of weight for the person when the person is stationary and/or during gait. The balance distribution of weight for the person may be measured when they are stationary using the smart floor tiles 112 by measuring the pressure applied to the smart floor tiles 112 by the left foot and right foot. If the balance distribution of weight changes by a threshold amount while stationary, it may indicate that the person is going to experience a fall event. Further, the balance distribution of weight for the person may be measure as the person gaits by measuring the pressure applied by the left foot and the right foot to the smart floor tiles 112. If the balance distribution of weight changes for the left foot or the right foot, that may indicate the person is swaying and is losing their balance and is likely to experience a fall event.

In some embodiments, historical information may be referenced that indicates people having certain physical measurements (e.g., height, weight, etc.) at certain ages typically have certain balance distribution of weight while stationary and during gait. In such an embodiment, gait baseline parameters may not be used and the historical information may be used to determine whether balance distribution of weights for people with similar physical measurements and age match are different by a threshold amount. If the balance distribution of weights differ by the threshold amount, then the person is likely to experience a fall event.

A twelfth parameter 924 may include a neurological condition of the person. Certain neurological conditions indicate a propensity for a fall event. For example, epilepsy, alzheimers, etc. may increase the chances of a person experiencing a fall event.

A thirteenth parameter 926 may include a change in stride of the person. Reduction in the length of stride of the person may indicate a propensity for a fall event. Also, reduction in stride time may indicate a propensity for the fall event.

A fourteenth parameter 928 may include a results of a calibration test. The calibration test may include the computer vision test, the smart floor tile test, and/or the moulding section test.

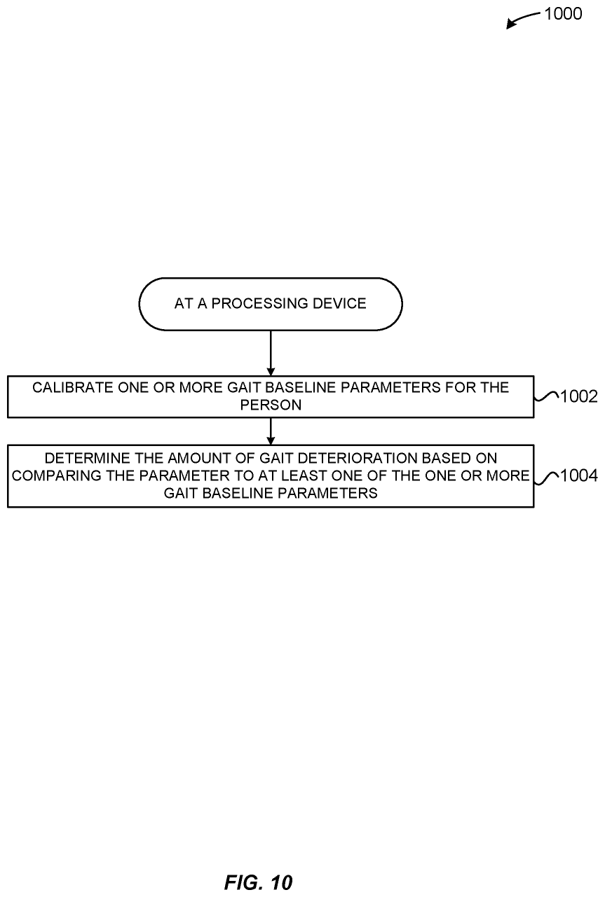

FIG. 10 illustrates an example of a method 1000 for using gait baseline parameters to determine an amount of gait deterioration according to certain embodiments of this disclosure. The method 1000 may be performed by processing logic that may include hardware (circuitry, dedicated logic, etc.), software, or a combination of both. The method 1000 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1000. The method 1000 may be implemented as computer instructions stored on a memory device and executable by the one or more processors. In certain implementations, the method 1000 may be performed by a single processing thread. Alternatively, the method 1000 may be performed by two or more processing threads, each thread implementing one or more individual functions, routines, subroutines, or operations of the methods.

At block 1002, the processing device may calibrate one or more gait baseline parameters for the person. Each gait baseline parameter may correspond with a separate respective parameter 900 that is monitored by the cloud-based computing system 116. The one or more gait baseline parameters may be stored in the database 129.

At block 1004, the processing device may determine the amount of gait deterioration based on comparing the parameter to at least one of the one or more gait baseline parameters. If the parameter varies by a certain amount or by the certain amount with a threshold period of time, then a certain amount of gait deterioration may be determined.

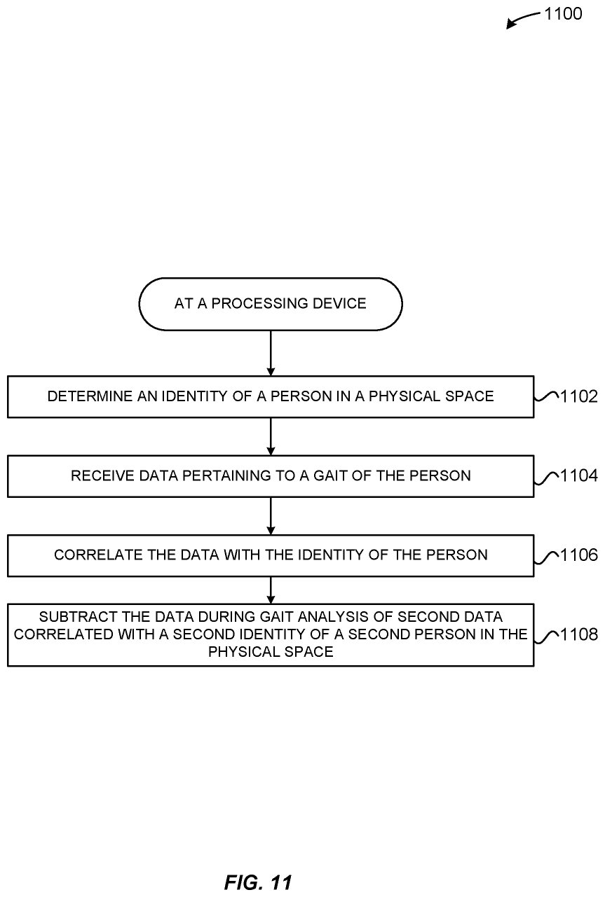

FIG. 11 illustrates an example of a method for subtracting data associated with certain people from gait analysis according to certain embodiments of this disclosure. The method 1100 may be performed by processing logic that may include hardware (circuitry, dedicated logic, etc.), software, or a combination of both. The method 1100 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1100. The method 1100 may be implemented as computer instructions stored on a memory device and executable by the one or more processors. In certain implementations, the method 1100 may be performed by a single processing thread. Alternatively, the method 1100 may be performed by two or more processing threads, each thread implementing one or more individual functions, routines, subroutines, or operations of the methods.

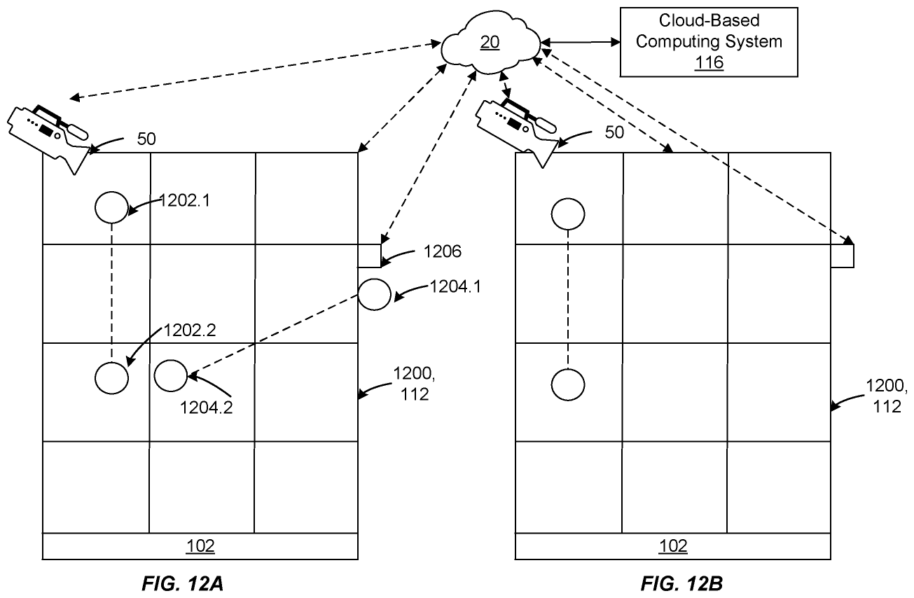

For purposes of clarity, FIGS. 11 and 12A-B are disclosed together below. FIG. 12A-B illustrate an overhead view of an example for subtracting data associated with certain people from gait analysis according to certain embodiments of this disclosure. Each square 1200 in FIGS. 12A-B represent a smart floor tile 112.

At block 1102, the processing device may determine an identity of a person (e.g., a medical personnel) in a physical space (e.g., a care room in a care facility where an elderly person is located). For example, the person may scan and/or swipe an identity badge at a reader 1206 disposed at an entry way (e.g., door) of the physical space in FIG. 12A. The data read by the reader 1206 may include the identity of the person, a user identification number, a job title, and the like. The data read may be transmitted by the reader 1206 to the cloud-based computing system 116. In some embodiments, the reader 1206 may be a camera and may be capable of performing facial recognition techniques on an image of the person to determine the identity of the person and/or transmit an image of the person to the cloud-based computing system 116 that is capable of performing facial recognition techniques on the image to determine the identity of the person.

At block 1104, the processing device may receive data pertaining to a gait of the person. The person may walk from a first position 1204.1 to a second position 1204.2 as depicted in FIG. 12A. The path of the person may be tracked based on data received via the smart floor tiles 112, the camera 50, and/or the moulding sections 102.

At block 1106, the processing device may correlate the data with the identity of the person. The correlated data with the identity of the person may be stored in the database 129.

At block 1108, the processing device may subtract the data during gait analysis of second data correlated with a second identity of a second person (e.g., an elderly person) in the physical space. For example, the person may walk from a first position 1202.1 to a second position 1202.2 in FIG. 12A. It may be desirable to just analyze the path of the person who may be a target person (e.g., elderly person in a care facility) and not the path of the medical personnel (e.g., nurse) entering the room. Subtracting the data correlated with the identity of the first person removes that data from the gait analysis of the second data correlated with the second identity of the second person, as depicted in FIG. 12B.

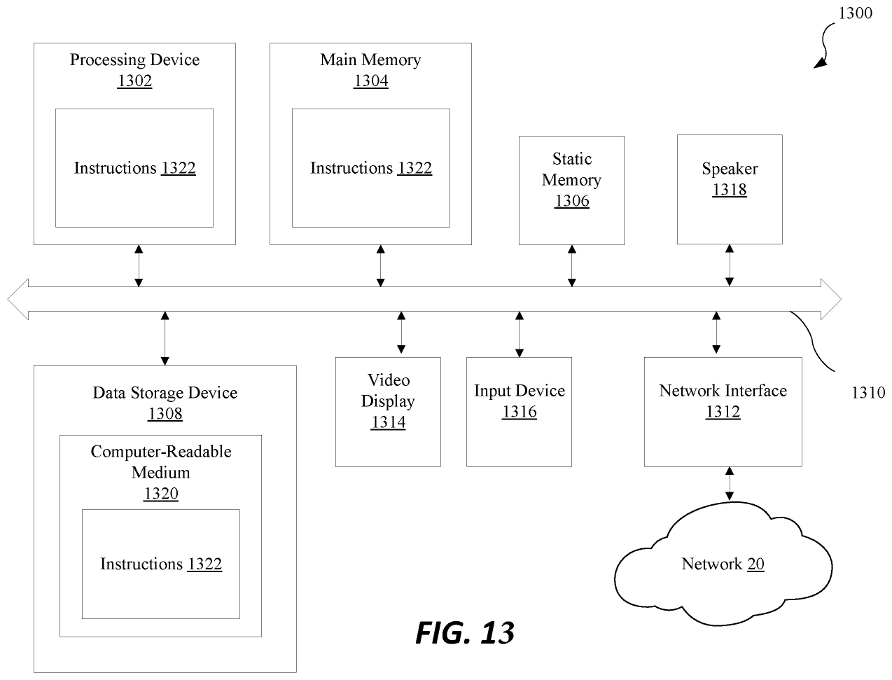

FIG. 13 illustrates an example computer system 1300, which can perform any one or more of the methods described herein. In one example, computer system 1300 may include one or more components that correspond to the computing device 12, the computing device 15, one or more servers 128 of the cloud-based computing system 116, the electronic device 13, the camera 50, the moulding section 102, the smart floor tile 112, or one or more training engines 152 of the cloud-based computing system 116 of FIG. 1B. The computer system 1300 may be connected (e.g., networked) to other computer systems in a LAN, an intranet, an extranet, or the Internet. The computer system 1300 may operate in the capacity of a server in a client-server network environment. The computer system 1300 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Some or all of the components computer system 1300 may be included in the camera 50, the moulding section 102, and/or the smart floor tile 112. Further, while only a single computer system is illustrated, the term “computer” shall also be taken to include any collection of computers that individually or jointly execute a set (or multiple sets) of instructions to perform any one or more of the methods discussed herein.

The computer system 1300 includes a processing device 1302, a main memory 1304 (e.g., read-only memory (ROM), solid state drive (SSD), flash memory, dynamic random access memory (DRAM) such as synchronous DRAM (SDRAM)), a static memory 1306 (e.g., solid state drive (SSD), flash memory, static random access memory (SRAM)), and a data storage device 1308, which communicate with each other via a bus 1310.

Processing device 1302 represents one or more general-purpose processing devices such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1302 may also be one or more special-purpose processing devices such as an application specific integrated circuit (ASIC), a field programmable gate array (FPGA), a digital signal processor (DSP), network processor, or the like. The processing device 1302 is configured to execute instructions for performing any of the operations and steps discussed herein.

The computer system 1300 may further include a network interface device 1312. The computer system 1300 also may include a video display 1314 (e.g., a liquid crystal display (LCD) or a cathode ray tube (CRT)), one or more input devices 1316 (e.g., a keyboard and/or a mouse), and one or more speakers 1318 (e.g., a speaker). In one illustrative example, the video display 1314 and the input device(s) 1316 may be combined into a single component or device (e.g., an LCD touch screen).

The data storage device 1316 may include a computer-readable medium 1320 on which the instructions 1322 embodying any one or more of the methodologies or functions described herein are stored. The instructions 1322 may also reside, completely or at least partially, within the main memory 1304 and/or within the processing device 1302 during execution thereof by the computer system 1300. As such, the main memory 1304 and the processing device 1302 also constitute computer-readable media. The instructions 1322 may further be transmitted or received over a network via the network interface device 1312.

While the computer-readable storage medium 1320 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The various aspects, embodiments, implementations or features of the described embodiments can be used separately or in any combination. The embodiments disclosed herein are modular in nature and can be used in conjunction with or coupled to other embodiments, including both statically-based and dynamically-based equipment. In addition, the embodiments disclosed herein can employ selected equipment such that they can identify individual users and auto-calibrate threshold multiple-of-body-weight targets, as well as other individualized parameters, for individual users.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

1. A method for determining a propensity for a fall event to occur, the method comprising:
receiving data from a sensing device in a smart floor tile;
monitoring a parameter pertaining to a gait of a person based on the data;
determining an amount of gait deterioration based on the parameter; and
determining whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.

2. The method of claim 1, wherein responsive to determining the propensity for the fall event for the person satisfies the threshold propensity condition, the method further comprises:
determining an intervention to perform based on the propensity for the fall event, and
performing the intervention.

3. The method of claim 2, wherein the intervention comprises:
transmitting a first message to a computing device of the person,
transmitting a second message to a computing device of a medical personnel,
causing an alarm to be triggered in a facility in which the person is located,
changing a property of an electronic device located in a physical space with the person,
changing a care plan for the person,
changing an intensity of a directional indicator in the physical space in which the person is located, or
some combination thereof.

4. The method of claim 1, wherein responsive to determining the propensity for the fall event for the person does not satisfy the threshold propensity condition, the method further comprises:
receiving subsequent data from the sensing device;
monitoring the parameter pertaining to the gait of the person based on the subsequent data;
determining a second amount of gait deterioration based on the parameter; and
determining whether the propensity for the fall event for the person satisfies the threshold propensity condition based on (i) the second amount of gait deterioration satisfying the threshold deterioration condition, (ii) the second amount of gait deterioration satisfying the threshold deterioration condition within the threshold time period, or some combination thereof.

5. The method of claim 1, wherein responsive to determining the propensity for the fall event for the person satisfies the threshold propensity condition, the method further comprises:
performing a type of intervention that has a severity that corresponds to the propensity for the fall event, the intervention included in a plurality of interventions that escalate in severity based on the propensity for the fall event.

6. The method of claim 1, wherein the parameter comprises at least one of:
a speed of the gait of the person,
a distance between a head of the person and feet of the person,
a distance between the feet during the gait of the person,
historical information pertaining to whether the person has previously fallen,
a weight of the person,
an age of the person,
medical history of the person,
fracture history of the person,
vision impairment of the person,
activity level of the person,
balance distribution of weight while stationary, during gait, or both,
neurological condition of the person,
change in stride of the person,
results of a calibration test, or
some combination thereof.

7. The method of claim 1, further comprising receiving the data from a camera, and wherein the parameter is monitored using computer vision, object recognition, measured pressure, location of feet of the person, or some combination thereof.

8. The method of claim 1, wherein the monitoring the parameter pertaining to the gait of the person based on the data, the determining the amount of gait deterioration based on the parameter, and the determining whether the propensity for the fall event for the person satisfies the threshold propensity condition further comprises:
inputting the data into one or more machine learning models trained to determine the amount of gait deterioration based on the parameter and to determine whether the propensity for the fall event for the person satisfies the threshold propensity condition.

9. The method of claim 8, wherein the one or more machine learning models comprise:
a first machine learning model trained to identify a change in the parameter and determine a first amount of gait deterioration,
a second machine learning model trained to identify a change in a second parameter pertaining to the gait of the person based on the data and determine a second amount of gait deterioration, and
a third machine learning model trained to:
determine the amount gate deterioration based on the first amount of gait deterioration and the second amount of gait deterioration, and
determine whether the propensity for the fall event for the person satisfies the threshold propensity condition based on (i) the amount of gait deterioration satisfying the threshold deterioration condition, (ii) the amount of gait deterioration satisfying the threshold deterioration condition within the threshold time period, or some combination thereof.

10. The method of claim 1, further comprising:
calibrating one or more gait baseline parameters for the person; and
determining the amount of gait deterioration based on comparing the parameter to at least one of the one or more gait baseline parameters.

11. A tangible, non-transitory computer-readable medium storing instructions that, when executed, cause a processing device to:
receive data from a sensing device in a smart floor tile;
monitor a parameter pertaining to a gait of a person based on the data;
determine an amount of gait deterioration based on the parameter; and
determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.

12. The computer-readable medium of claim 11, wherein responsive to determining the propensity for the fall event for the person satisfies the threshold propensity condition, the processing device is further to:
determine an intervention to perform based on the propensity for the fall event, and
perform the intervention.

13. The computer-readable medium of claim 12, wherein the intervention comprises:
transmitting a first message to a computing device of the person,
transmitting a second message to a computing device of a medical personnel,
causing an alarm to be triggered in a facility in which the person is located,
changing a property of an electronic device located in a physical space with the person,
changing a care plan for the person,
changing an intensity of a directional indicator in the physical space in which the person is located, or
some combination thereof.

14. The computer-readable medium of claim 11, wherein responsive to determining the propensity for the fall event for the person does not satisfy the threshold propensity condition, the processing device is further to:
receive subsequent data from the sensing device;
monitor the parameter pertaining to the gait of the person based on the subsequent data;
determine a second amount of gait deterioration based on the parameter; and
determine whether the propensity for the fall event for the person satisfies the threshold propensity condition based on (i) the second amount of gait deterioration satisfying the threshold deterioration condition, (ii) the second amount of gait deterioration satisfying the threshold deterioration condition within the threshold time period, or some combination thereof.

15. The computer-readable medium of claim 11, wherein responsive to determining the propensity for the fall event for the person satisfies the threshold propensity condition, the processing device is further to:
perform a type of intervention that has a level that corresponds to the propensity for the fall event, the intervention included in a plurality of interventions that escalate in severity based on how imminent the fall event is to occurring determined by the propensity for the fall event.

16. The computer-readable medium of claim 11, wherein the parameter comprises:
a speed of the gait of the person,
a distance between a head of the person and feet of the person,
a distance between the feet during the gait of the person,
historical information pertaining to whether the person has previously fallen,
a weight of the person,
an age of the person,
medical history of the person,
fracture history of the person,
vision impairment of the person,
activity level of the person,
balance distribution of weight while stationary, during gait, or both,
neurological condition of the person,
change in stride of the person,
results of a calibration test, or
some combination thereof.

17. The computer-readable medium of claim 11, wherein the processing device is further to receive the data from a camera, and the parameter is monitored using computer vision, object recognition, measured pressure, location of feet of the person, or some combination thereof.

18. The computer-readable medium of claim 11, wherein to monitor the parameter pertaining to the gait of the person based on the data, determine the amount of gait deterioration based on the parameter, and determine whether the propensity for the fall event for the person satisfies the threshold propensity condition, the processing device is further to:
input the data into one or more machine learning models trained to determine the amount of gait deterioration based on the parameter and to determine whether the propensity for the fall event for the person satisfies the threshold propensity condition.

19. The computer-readable medium of claim 18, wherein the one or more machine learning models comprise:
a first machine learning model trained to identify a change in the parameter and determine a first amount of gait deterioration,
a second machine learning model trained to identify a change in a second parameter pertaining to the gait of the person based on the data and determine a second amount of gait deterioration, and
a third machine learning model trained to:
determine the amount gate deterioration based on the first amount of gait deterioration and the second amount of gait deterioration, and
determine whether the propensity for the fall event for the person satisfies the threshold propensity condition based on (i) the amount of gait deterioration satisfying the threshold deterioration condition, (ii) the amount of gait deterioration satisfying the threshold deterioration condition within the threshold time period, or some combination thereof.

20. A system comprising:
a memory device storing instructions;
a processing device communicatively coupled to the memory device, the processing device to execute the instructions to:
receive data from a sensing device in a smart floor tile;
monitor a parameter pertaining to a gait of a person based on the data;
determine an amount of gait deterioration based on the parameter; and
determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period.
